(19) United States
(12) Patent Application Publication
(10) Pub. No.: US 2019/0357853 A1
(43) Pub. Date: Nov. 28, 2019

(54) DIABETES RISK ENGINE AND METHODS THEREOF FOR PREDICTING DIABETES PROGRESSION AND MORTALITY

(71) Applicants: Lizheng Shi, New Orleans, LA (US); Vivian A. Fonseca, New Orleans, LA (US); Hui Shao, Dunwoody, GA (US)

(72) Inventors: Lizheng Shi, New Orleans, LA (US); Vivian A. Fonseca, New Orleans, LA (US); Hui Shao, Dunwoody, GA (US)

(21) Appl. No.: 16/422,934

(22) Filed: May 24, 2019

Related U.S. Application Data
(60) Provisional application No. 62/676,273, filed on May 24, 2018.

(51) Int. Cl. A61B 5/00; G16H 50/30; G16H 50/20; G16H 50/50

(52) U.S. Cl. CPC A61B 5/7275; G16H 50/30; A61B 5/4842; G16H 50/50; A61B 5/742; G16H 50/20

(57) ABSTRACT

The present disclosure provides for diabetes risk engine systems and methods for predicting diabetes progression and mortality in a patient with type 2 diabetes mellitus, for the U.S. population, including the building, relating, assessing, and validating outcomes (BRAVO) risk engine. The BRAVO risk engine includes a diabetes-related events module to predict an occurrence of one or more events, a risk factors module to predict a progression of risk factors, a mortality module to predict an occurrence of mortality, and a display interface configured to display the predicted risk of diabetes-related events or mortality. Risk equations for predicting diabetes-related microvascular and macrovascular events, hypoglycemia, mortality, and progression of diabetes risk factors were estimated using the data from the Action to Control Cardiovascular Risk in Diabetes (ACCORD) trial. The BRAVO risk engine preferably includes risk factors including severe hypoglycemia and common U.S. racial/ethnicity categories, compared to the UKPDS risk engine.

### CROSS-REFERENCE TO RELATED APPLICATIONS

This application claims the benefit of priority of U.S. Provisional Application No. 62/676,273, filed May 24, 2018, the disclosure of which is hereby incorporated by reference in its entirety.

### BACKGROUND OF THE INVENTION

### Field of the Invention

The present invention generally relates to risk engines for disease management and more particularly relates to diabetes risk engine systems and methods for predicting diabetes progression, mortality, and generating recommendations and actions for healthcare advice for general improvement of patients.

### Description of the Related Art

The growing population of type 2 diabetes mellitus (T2DM) in the United States and abroad has led to dramatically increased costs in managing diabetes, including treating diabetes and its complications. A majority of the diabetes-related costs are the result of micro/macrovascular complication events. The most common diabetes-related macrovascular events include myocardial infarction (MI), congestive heart failure (CHF), and stroke. The most frequent diabetes-related microvascular events include AQ3 retinopathy (e.g., edema, blindness), nephropathy [e.g., end-stage renal disease (ESRD)], and neuropathy [e.g., severe pressure sensation loss (SPSL), amputation].

To better manage the growing T2DM population in an environment of constrained healthcare resources, there is a need for system-wide improvement and redesign, which in the current ‘big data’ era, is made possible through embodiments disclosed herein, using outcome-driven and evidence-based diabetes management. As disclosed herein, prediction models can help develop sophisticated and well-designed diabetes management strategies and models. Prediction models, disclosed herein, can better profile the risk of patients so that more healthcare resources can be effectively allocated to those with more health needs.

Several conventional diabetes models in the United States have been used to describe disease progression and compare the cost effectiveness of different therapeutic strategies, such as, the CORE diabetes model, the University of Michigan model for diabetes, the Swedish Institute of Health Economics model, otherwise known as the Economics and Health Outcomes in T2DM Model, the United Kingdom Prospective Diabetes Study (UKPDS) outcomes model, the Centers for Disease Control-Research Triangle Institute diabetes cost-effectiveness model, the Cardiff Research Consortium model, and several others. These conventional models have been used to support the outcome-driven evidence-based diabetes management in several areas, such as, comparisons between therapeutic plans, evaluating potential benefits of achieving treatment goals, and policy impact on T2DM. However, these diabetes models rely heavily on the UKPDS risk engine and Framingham equation that was developed using data from a UK diabetes cohort collected from the 1970s of European populations. The UKPDS population differs significantly from the current U.S. population in terms of race/ethnicity, definition of diabetes, treatment algorithm, and screening methods to assess complications and comorbidities. Further, the baseline hazard of diabetes-related events may vary over time and may differ between the UKPDS population and the current U.S. population. Using a UK-based risk engine to predict U.S. diabetes management raises significant concerns on the prediction validity.

While these models may be moderately suitable for the particular purpose employed, they would not be as suitable for the purposes of the present invention as disclosed hereafter.

Accordingly, there is an urgent need for new and improved risk engines that are developed based on a U.S. population, to better support decision making in clinical practice in the United States.

There is a need for new and improved diabetes modeling that incorporates new data and that can be tailored to changing national priorities in the prevention and treatment of diabetes.

Assessing the risks and progression of diseases can be useful in disease prevention and mitigation.

In light of the discussion above, there is a need for new computer implemented methods and systems for predicting diabetes progression and mortality, which alleviates one or more of the above mentioned deficiencies.

Therefore, one object of the invention is to provide for a Building, Relating, Assessing, and Validating Outcomes (BRAVO) Diabetes Risk Engine based on the U.S. diabetes population, which provides an alternative risk engine for U.S. researchers and policy makers.

It is yet another object of the invention to provide a critical predictive modeling tool to evaluate new T2DM drugs.

It is yet a further object of the invention to provide a diabetes model to include race segmentation relevant to the target population for clinical intervention.

Another object of the invention is to provide a novel approach to using clinical trials with a limited length of follow-up time.

Through embodiments disclosed herein, the BRAVO risk engine provides more accurate predictions over a range of long-term outcomes as opposed to other current models. Thus, the BRAVO risk engine provides substantially improved assistance in making clinical and policy decisions.

As the most commonly used regression class to model the risk of clinical events, the parametric proportional hazard function was applied in the BRAVO risk engine. The main reason for researchers not being able to update the UKPDS risk engine is the data limitation issue. To fit prediction models for a lifetime disease progression, a clinical trial with more than 30 years follow-up time was required. There are no other clinical trial with such a length of follow up as UKPDS trial. However, even if there was one, by the time the 30 years follow-up data would be collected, the data itself would become outdated.

The BRAVO risk engine uses diabetes duration as a time index to simulate diabetes progression and mortality over a period of 40 years, in accordance with embodiments of the invention. Such embodiments of indexing time by diabetes duration enables one to estimate the time dependency of diabetes on events and mortality.

As disclosed in this application, the inventor has discovered novel and unique systems and methods for efficient and comprehensive prediction of diabetes progression and mortality, which exhibit superlative properties.

Embodiments of the present invention provide for systems and methods, as disclosed herein, defined in the annexed claims which provide for an improved risk engine that can predict a range of long-term diabetes complications and mortality, thus assisting in making clinical and policy decisions for people's health and well-being.

### SUMMARY OF THE INVENTION

The following presents a simplified summary of the present disclosure in a simplified form as a prelude to the more detailed description that is presented herein.

Therefore, in accordance with embodiments of the invention, there is provided a diabetes risk engine system having at least one processor, at least one memory unit containing computer program code, a diabetes-related events module configured to predict an occurrence of one or more diabetes-related events through an iterative process, a risk factors module to predict a progression of one or more risk factors through the iterative process, a mortality module to predict an occurrence of mortality of a patient through the iterative process, and a display interface configured to display the predicted risk of the one or more diabetes-related events.

In one embodiment, the risk factor is selected from the group consisting of glycosylated hemoglobin (HbA1c), systolic blood pressure (SBP), weight, and low-density lipoprotein cholesterol (LDL-C).

In another embodiment, the diabetes-related event is a macrovascular event. Exemplary macrovascular events include stroke, myocardial infarction, congestive heart failure, angina, and revascularization surgery.

In yet another embodiment, the diabetes-related event is a microvascular event. Exemplary microvascular events include end stage renal failure, blindness, and severe pressure sensation loss.

In one embodiment, the diabetes-related event is an adverse event. Exemplary adverse events include severe hypoglycemia and symptomatic hypoglycemia.

The ACCORD dataset used for the BRAVO risk engine features: a record of diabetes duration for each patient and patients' diabetes duration varied from 0 years to 40 years in the dataset. Regardless of the maximum 7 years follow up length for this clinical trial, the invention has detailed records for the incidence of events, as well as the risk factors and history of events at each time point after diabetes onset. This feature enables the BRAVO risk engine to more accurately estimate the hazard rates at each time point after diabetes onset, and a left-truncated survival regression applied to estimate the prediction equations of each diabetes related event.

Therefore, in accordance with embodiments of the invention, there is provided a method of predicting diabetes progression and mortality. The method includes a first step of receiving a target population dataset having a series of baseline biological characteristics of the target population. The method has a second step of assigning parameter values based upon a user defined distribution of population characteristics. A third step analyzes, using a computer processor, the population dataset for generating a diabetes risk engine wherein diabetes duration is used as a time index. The diabetes risk engine operates one or more inter-correlated risk equations which can be used as a predictor to determine the risk of a diabetes-related event or mortality of the patient. A fourth step of the method adjusts the risk engine based upon new data collected based upon a new set of annual values collected during the generating of the population dataset. A fifth step of the method receives a first patient medical information, which includes clinical, biomedical, and demographic factor information. A sixth step of the method predicts a risk of an occurrence of one or more diabetes-related events with the diabetes risk engine based upon said parameter values by comparing the first patient medical information to the population dataset. A seventh step of the method provides at least one clinical or behavioral modification recommendation based upon the predicted risk of the occurrence of one or more diabetes-related events or mortality. An eighth step of the method tracks a progression and survival status of a user taking action toward achieving goals associated with improving health based upon at least one clinical or behavioral modification recommendation.

In one embodiment, the method further includes a step of predicting a progression of risk factors of the first patient.

In another embodiment, the inter-correlated risk equations account for risk escalation as diabetes progresses and during interactions between complications.

In yet another embodiment, the first patient medical information includes risk factors such as medication adherence, lifestyle modification, and therapy escalation.

In one embodiment, the diabetes-related event is a macrovascular event. Exemplary macrovascular events include stroke, a myocardial infarction, congestive heart failure, angina, and revascularization surgery.

In another embodiment, the diabetes-related event is a microvascular event. Exemplary macrovascular events include end stage renal failure, blindness, and severe pressure sensation loss.

In yet another embodiment, the diabetes-related event is an adverse event. Exemplary adverse events include hypoglycemia and symptomatic hypoglycemia.

In accordance with embodiments of the invention, there is provided a method of predicting an occurrence of one or more diabetes-related events having the first step of receiving a target population dataset comprising a series of baseline characteristics of the target population. A second step of the method assigns parameter values based upon a user defined distribution of population characteristics. A third step of the method includes analyzing, using a computer processor, the population dataset for generating a diabetes risk engine wherein diabetes duration is used as a time index. In the third step, the diabetes risk engine operates one or more inter-correlated risk equations configured to determine the risk of a diabetes-related event or mortality of the patient. A fourth step of the method adjusts the risk engine based upon new data collected based upon a new set of annual values collected during the generating of the population dataset. A fifth step of the method receives a first patient medical information such as clinical, biomedical, and demographic factor information from a first patient user. A sixth step of the method predicts a risk of an occurrence of one or more diabetes-related events with the diabetes risk engine based upon the parameter values by comparing the first patient medical information to the population dataset of the diabetes risk engine. A seventh step of the method provides at least one clinical or behavioral modification recommendation based on the risk of the occurrence of one or more diabetes-related events. An eighth step tracks the survival status of a user taking action toward achieving goals associated with improving health based upon the at least one clinical or behavioral modification recommendation.

These and other features, aspects, and advantages of the present invention will become better understood with reference to the following description and appended claims.

### BRIEF DESCRIPTION OF THE DRAWINGS

Illustrative embodiments of the present invention are described herein with reference to the accompanying drawings, in which like numerals throughout the figures identify substantially similar components, in which:

FIG. 1 is a schematic of an exemplary embodiment of a diabetes risk engine system and environment, in accordance with embodiments of the invention;

FIG. 2 is the functional form of risk equations and model selection processes, in accordance with embodiments of the invention;

FIG. 3 is a flowchart for microsimulation, including a flow control engine and a risk engine, in accordance with embodiments of the invention;

FIG. 4 is a table displaying baseline characteristics of the ACCORD cohort, in accordance with embodiments of the invention;

FIG. 5 is a table displaying variables included in the risk engine, in accordance with embodiments of the invention;

FIG. 6 is a table displaying prediction equations for time varying risk factors, in accordance with embodiments of the invention;

FIG. 7 is a table displaying differences between RECODE risk equations and BRAVO risk equations, in accordance with embodiments of the invention;

FIG. 8 is a table displaying parameter estimates of risk equations in the BRAVO risk engine, in accordance with embodiments of the invention;

FIG. 9 is an exemplary flowchart illustrating an exemplary method of implementing the BRAVO risk engine, in accordance with embodiments of the invention;

FIG. 10 is an exemplary flowchart illustrating an exemplary method of implementing the BRAVO risk engine, in accordance with embodiments of the invention;

FIG. 11 is a graphic display of exemplary external validation results plotting predicted incident rates against observed incidence rates of CHF (congestive heart failure), CVD (cardiovascular disease), and MI (myocardial infarction), in accordance with embodiments of the invention;

FIG. 12 is a table displaying definitions of variables included in the risk engine, in accordance with embodiments of the invention;

FIG. 13 is a table displaying exemplary demographic characteristics of the ACCORD cohort, in accordance with embodiments of the invention;

FIG. 14 is a table displaying an exemplary prediction equation for stroke events, in accordance with embodiments of the invention;

FIG. 15 is a graphic display of the predicted versus observed cumulative incidence for stroke events according to diabetes duration, in accordance with embodiments of the invention;

FIG. 16 is a table displaying an exemplary prediction equation for CHF, in accordance with embodiments of the invention;

FIG. 17 is a graphic display of the predicted versus observed cumulative incidence for CHF according to diabetes duration, in accordance with embodiments of the invention;

FIG. 18 is a table displaying an exemplary prediction equation for MI, in accordance with embodiments of the invention;

FIG. 19 is a graphic display of the predicted versus observed cumulative incidence for non-fatal MI according to diabetes duration, in accordance with embodiments of the invention;

FIG. 20 is a table displaying an exemplary prediction equation for angina, in accordance with embodiments of the invention;

FIG. 21 is a graphic display of the predicted versus observed cumulative incidence for angina according to diabetes duration, in accordance with embodiments of the invention;

FIG. 22 is a table displaying an exemplary prediction equation for revascularization surgery, in accordance with embodiments of the invention;

FIG. 23 is a graphic display of the predicted versus observed cumulative incidence for revascularization surgery according to diabetes duration, in accordance with embodiments of the invention;

FIG. 24 is a table displaying an exemplary prediction equation for ESRD, in accordance with embodiments of the invention;

FIG. 25 is a graphic display of the predicted versus observed cumulative incidence for ESRD according to diabetes duration, in accordance with embodiments of the invention;

FIG. 26 is a table displaying an exemplary prediction equation for blindness, in accordance with embodiments of the invention;

FIG. 27 is a graphic display of the predicted versus observed cumulative incidence for blindness according to diabetes duration, in accordance with embodiments of the invention;

FIG. 28 is a table displaying an exemplary prediction equation for SPSL, in accordance with embodiments of the invention;

FIG. 29 is a graphic display of the predicted versus observed cumulative incidence for SPSL according to diabetes duration, in accordance with embodiments of the invention;

FIG. 30 is a table displaying an exemplary prediction equation for all-cause mortality, in accordance with embodiments of the invention;

FIG. 31 is a graphic display of the predicted versus observed cumulative incidence for all-cause mortality according to age, in accordance with embodiments of the invention;

FIG. 32 is table displaying an exemplary prediction equation for CVD death (logistic regression), in accordance with embodiments of the invention;

FIG. 33 is a graphic display of the ROC curve for CVD prediction, in accordance with embodiments of the invention;

FIG. 34 is a graphic display of the predicted versus observed cumulative incidence for CVD death according to age, in accordance with embodiments of the invention;

FIG. 35 is a table displaying exemplary prediction equations for time varying risk factors, in accordance with embodiments of the invention;

FIG. 36 is a table displaying exemplary prediction equations for hypoglycemia, in accordance with embodiments of the invention;

FIG. 37 is a table displaying an exemplary prediction equation for smoking, in accordance with embodiments of the invention;

FIG. 38 is a graphic display of the ROC curve for smoking equation, in accordance with embodiments of the invention;

FIG. 39 is a table displaying the exemplary use of the UKPDS risk engine to predict the ACCORD cohort, in accordance with embodiments of the invention;

FIG. 40 is a table displaying exemplary baseline characteristics of the clinical trials used in external validation, in accordance with embodiments of the invention;

FIG. 41 is a table displaying the exemplary comparison of prediction accuracy across different models on the ASPEN cohort, in accordance with embodiments of the invention;

FIG. 42 is a table displaying the exemplary comparison of prediction accuracy across different models on the CARDS cohort, in accordance with embodiments of the invention;

FIG. 43 is a table displaying the exemplary comparison of prediction accuracy across different models on the ADVANCE cohort, in accordance with embodiments of the invention; and

FIG. 44 is a table displaying the exemplary comparison of prediction accuracy across different models on the ACCORD cohort, in accordance with embodiments of the invention.

### DETAILED DESCRIPTION

For a further understanding of the nature and function of the embodiments, reference should be made to the following detailed description. Detailed descriptions of the embodiments are provided herein, as well as the best mode of carrying out and employing the present invention. It will be readily appreciated that the embodiments of the invention are well adapted to carry out and obtain the ends and features mentioned, as well as those inherent herein. It is to be understood, however, that the present invention may be embodied in various forms. Therefore, persons of ordinary skill in the art will realize that the following disclosure is illustrative only and not in any way limiting, as the specific details disclosed herein provide a basis for the claims and a representative basis for teaching to employ the present invention in virtually any appropriately detailed system, structure or manner. It should be understood that the devices, materials, methods, procedures, and techniques described herein are presently representative of various embodiments. Other embodiments of the disclosure will readily suggest themselves to such skilled persons having the benefit of this disclosure.

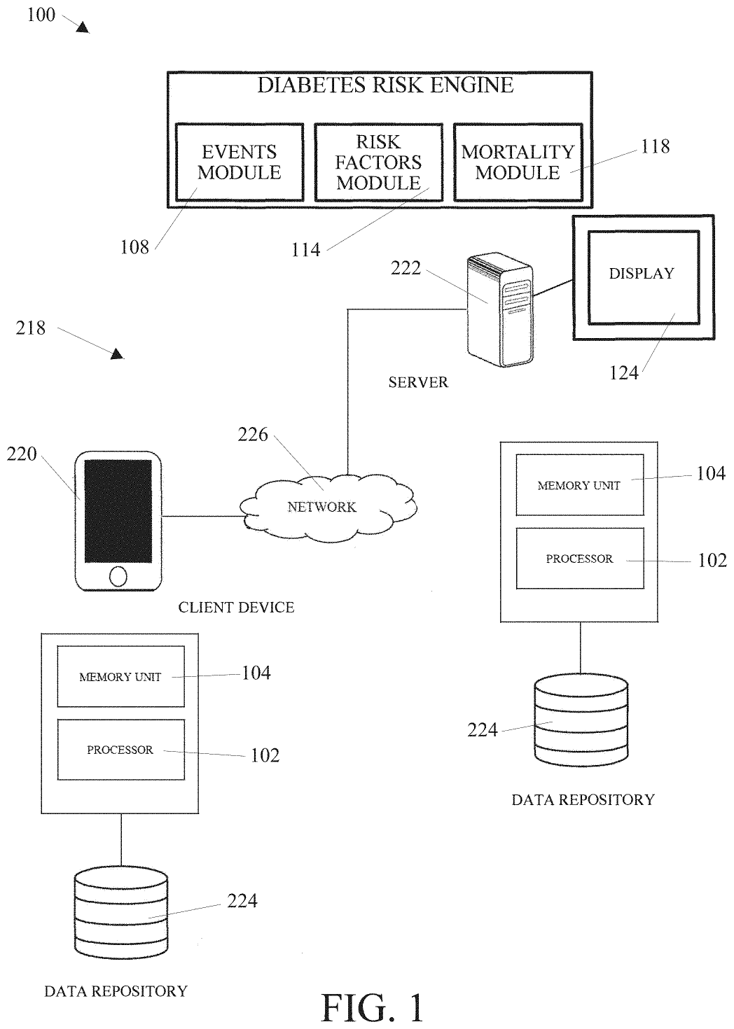

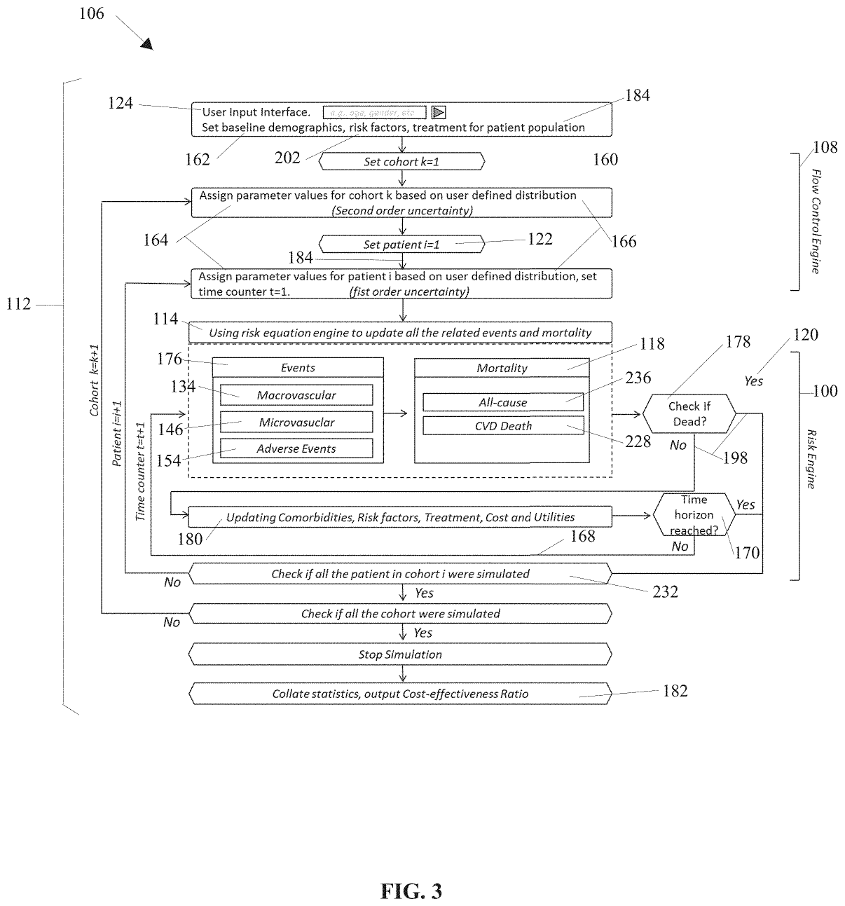

Therefore, in accordance with embodiments of the invention, there is provided a diabetes risk engine system 100 and an exemplary environment 218, which may include multiple devices. Referring initially to FIGS. 1 and 3, the Building, Relating, Assessing, and Validating Outcomes (BRAVO) risk engine 100 and the exemplary environment 218 have at least one processor 102, at least one memory unit 104 containing computer program code 106, at least one client user device 220, at least one application server or other device 222 for running the various engines described herein, and at least one additional server or database 224. The processor 102 is configured to execute software instructions 106 stored on a tangible, non-transitory computer readable storage medium 104 (e.g., hard drive, solid state drive, RAM, flash, ROM, etc.). The software instructions 106 preferably configure the computing device 220 to provide the roles, responsibilities, or other functionality as discussed herein with respect to the disclosed apparatus and methods. In such preferred embodiment, the various servers 222, systems, databases 224, or interfaces 220 exchange data using standardized protocols or algorithms 106, possibly based on HTTP, HTTPS, AES, public-private key exchanges, web service APIs, known financial transaction protocols, or other electronic information exchanging methods. Data exchanges preferably are conducted over a network 226 (e.g., packet-switched network, the Internet, LAN, WAN, VPN, or other type of packet switched network). Processors 102 can be embodied by any computational or data processing device 220, such as a central processing unit (CPU), application specific integrated circuit (ASIC), or comparable device. The processors 102 can be implemented as a single controller, or as a plurality of controllers or processors. For example, the processors 102 can be implemented using a single core chip or one or more multi-core chips, among other possible configurations.

The exemplary memories 104 can independently be any suitable storage device, such as a non-transitory computer-readable medium. A hard disk drive (HDD), random access memory (RAM), flash memory, or other suitable memory can be used. The memories 104 can be combined on a single integrated circuit as the processor 102, or may be separate from the one or more processors 102. Furthermore, the computer program instructions 106 stored in the memory 104, and which may be processed by the processors 102, can be any suitable form of computer program code 106; for example, a compiled or interpreted computer program written in any suitable programming language. The memory 104 and the computer program instructions 106 can be configured with the processor 102 for the particular device to cause a hardware apparatus, such as the user device 220, the application server 222, and additional servers or databases 224 to perform any of the processes described below. Therefore in certain embodiments, a non-transitory computer-readable medium 104 can be encoded with computer instructions 106 that when executed in hardware performs a process such as one of the processes described herein. Alternatively, certain embodiments of the invention can be performed entirely in hardware. The BRAVO risk engine 100 has been developed to predict 174 a series of diabetes complications 186 and mortality 178. The BRAVO risk engine 100 has found a glycosylated hemoglobin level 126 slightly above 7.0% to be associated with the lowest risk for all-cause mortality 236. With good internal and external validation, the BRAVO risk engine 100 can be applied as a diabetes prediction model 100 and assists in decision making for clinical practice 186 and health policy 188.

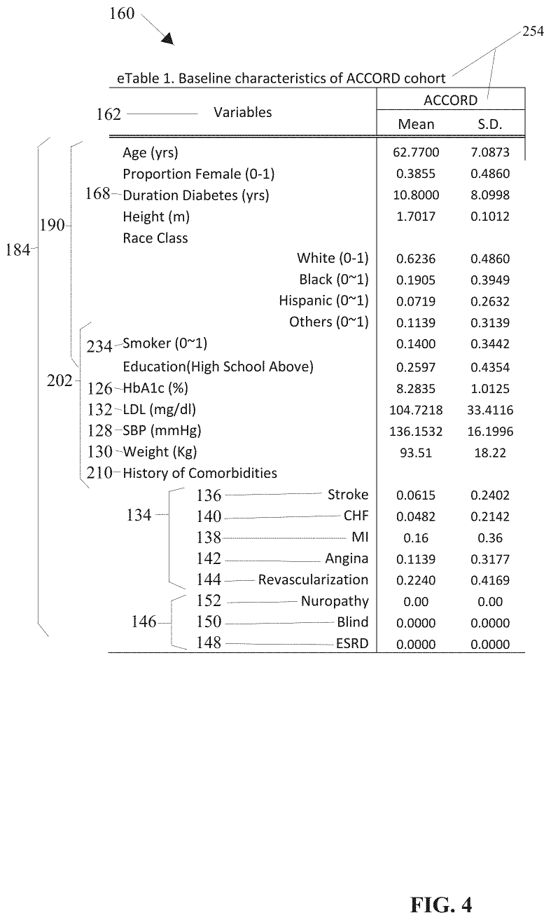

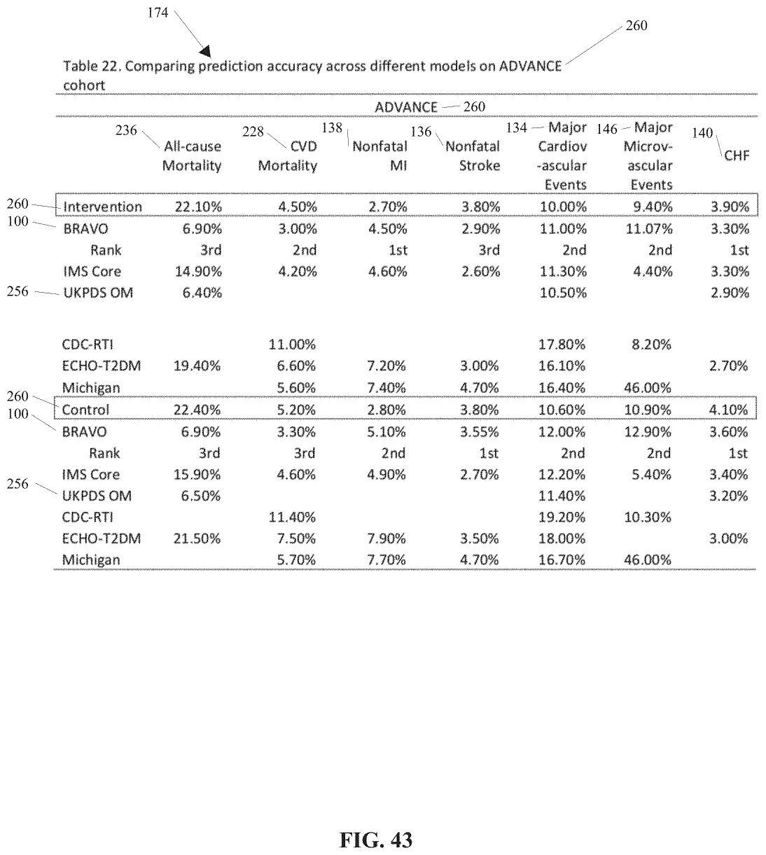

Referring to FIGS. 4, 11, and 40, when evaluating the prediction 174 accuracy of the BRAVO risk engine 100, especially to explore if it is capable of providing more accurate predictions 174 compared to the existing models, a prediction model was applied on ASPEN trials 258, ADVANCE trials 260 and CARDS trials 262. Because those three trials had been used as challenges in the Fourth and Fifth Mount Hood challenge meeting, in implementing the BRAVO risk engine 100, the prediction 174 results of each current model on those three trials were extracted and compared to simulation results of the BRAVO risk engine 100. The prediction accuracy of each model is outlined in FIG. 43.

The Atorvastatin Study for Prevention of Coronary Heart Disease Endpoints in Non-Insulin-Dependent Diabetes Mellitus (ASPEN) 258 was a 4-year, double-blind, parallel group trial of 10 mg of atorvastatin versus placebo inpatients with type 2 diabetes. The target LDL 132 level of the treatment group and control group was 83 mg/dl and 113 mg/dl, respectively. The provided outcomes included 4 years all-cause mortality 236 rate, CVD 228 mortality rate, nonfatal/fatal MI 138 incidence rate, nonfatal/fatal stroke 136 incidence rate, and angina 142 incidence rate.

The Action in Diabetes and Vascular Disease: Preterax and Diamicron Modified Release Controlled Evaluation (ADVANCE) 260 is a 5-year, double-blind trial, with a total of 11,140 T2DM patients 122 randomly assigned to either standard glucose control or intensive glucose control. The HbA1c 126 target for treatment group and control group was 6.5% and 7.5% respectively. The provided outcomes included 5-year all-cause mortality 236 rate, CVD 228 mortality rate, nonfatal MI 138 incidence rate, nonfatal stroke 136 incidence rate, major macrovascular events 134 incidence rate, major microvascular events 146 incidence rate, and CHF 140 incidence rate.

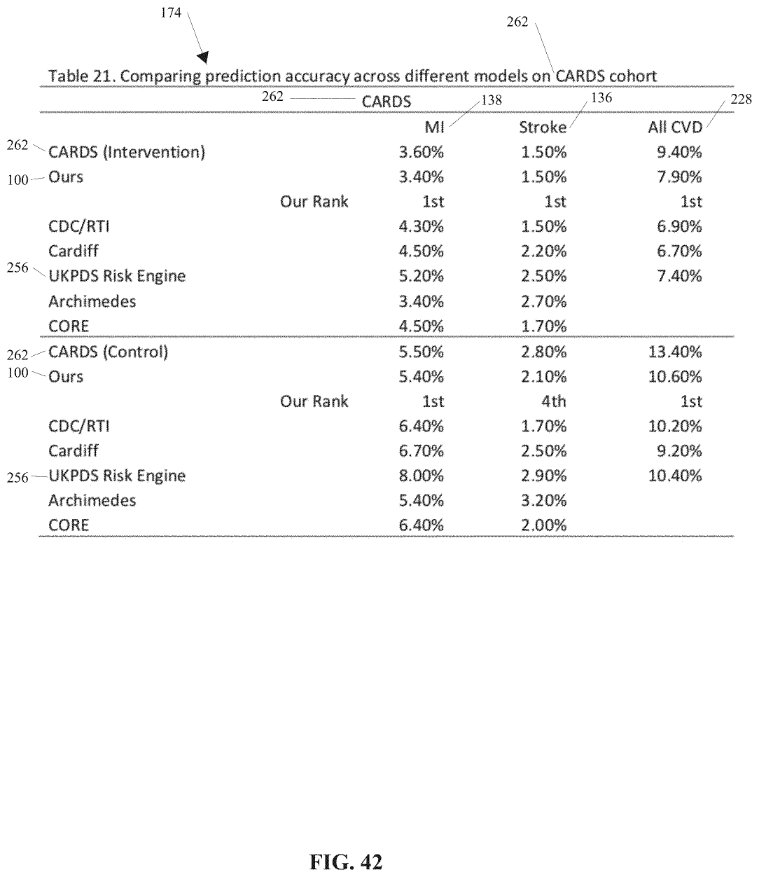

The Collaborative Atorvastatin Diabetes Study (CARDS) 262 is a 4 years, multicenter randomized placebo-controlled trial. The target LDL 132 level of treatment group and control group was 80 mg/dl and 120 mg/dl, respectively. The provided outcomes included 4-year fatal/nonfatal MI 138 incidence rate, fatal/nonfatal stroke 136 incidence rate, and all CVD 228 incidence rate. The prediction accuracy of CARDS 262 has been compared to the BRAVO risk engine's 100 prediction accuracy, as illustrated in FIG. 42.

The baseline characteristics 162 and treatment target 200 of each trial was input into the BRAVO risk engine 100, and end points for each trial was simulated and recorded. The predicted incidence rate 192 for each end point are compared to the observed incidence rate 232 in each trial. This embodiment also calculates the absolute difference between the predicted 192 and observed incidence rate 232, and compared if the absolute difference of the BRAVO risk engine 100 prediction 174 was smaller than the absolute difference provided by other models.

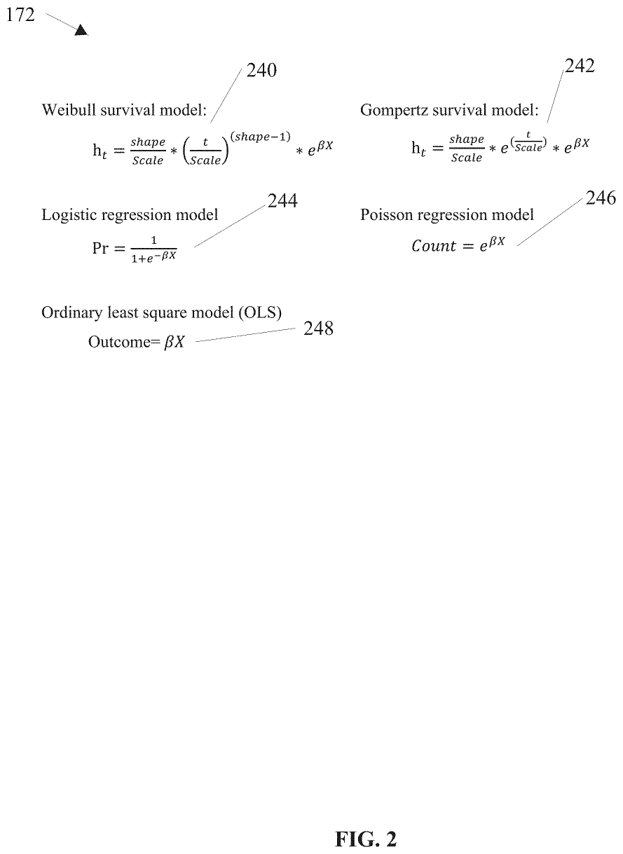

In such preferred embodiments, illustrated in FIGS. 1-3, the BRAVO risk engine 100 contains three separate modules (a diabetes-related events events module 108, a risk factors module 114, and a mortality module 118), each of which preferably contains a series of regression equations 172 to predict the occurrence of events 110, progression in risk factors 116, and mortality 178, as illustrated in FIG. 2. Risk factors progress 116 very differently under different circumstances, and largely depend on factors such as medication adherence 212, lifestyle modification 214, and therapy escalation 216. A few potential comorbidities such as ulceration and amputation were not explicitly included in the BRAVO risk engine 100. However, these comorbidities were not included as endpoints in the ACCORD trial 254, and thus cannot be incorporated into the BRAVO risk engine 100. Having said that, alternative embodiments may include these comorbidities. The diabetes-related events module 108 is configured to predict 174 an occurrence of one or more diabetes-related events 110 through an iterative process 112. The risk factors module 114 is configured to predict 174 a progression of one or more risk factors 116, which are based on one or more parameter values 164 and a set of predefined risk factors 202, through the iterative process 112. The mortality module 118 is configured to predict 174 an occurrence of mortality 120 of a patient 122 through the iterative process 112, and a display interface 124 is configured to display the predicted risk of the one or more diabetes-related events 176. In certain systems 100, only the user device 220 and application server 222 may be present, and in other systems 100 the user device 220, application server 222, and a plurality of other servers 224 may be present. Other configurations are also possible. The additional server or database 224 may be, for example, a database 224 securely storing one or more population datasets 160 comprising a series of baseline characteristics 162, medical records, facts, and recommendations 196. The at least one user device 220 can be, for example, a smartphone, tablet, portable computer, laptop computer, or other computing device. The user device 220 is preferably provided with a graphical user interface 124 and inputs, such as a touch-sensitive screen, display, keyboard, mouse, or the like. Other features are also permitted on the user device 220. The user device 220 can be a terminal device. Preferably, the user interface 124 is constructed based on Visual Basic Application (VBA) in Excel 2013 and the risk engine 100 is coded with C++ via Microsoft Visual Studio 15.0 Enterprise Edition.

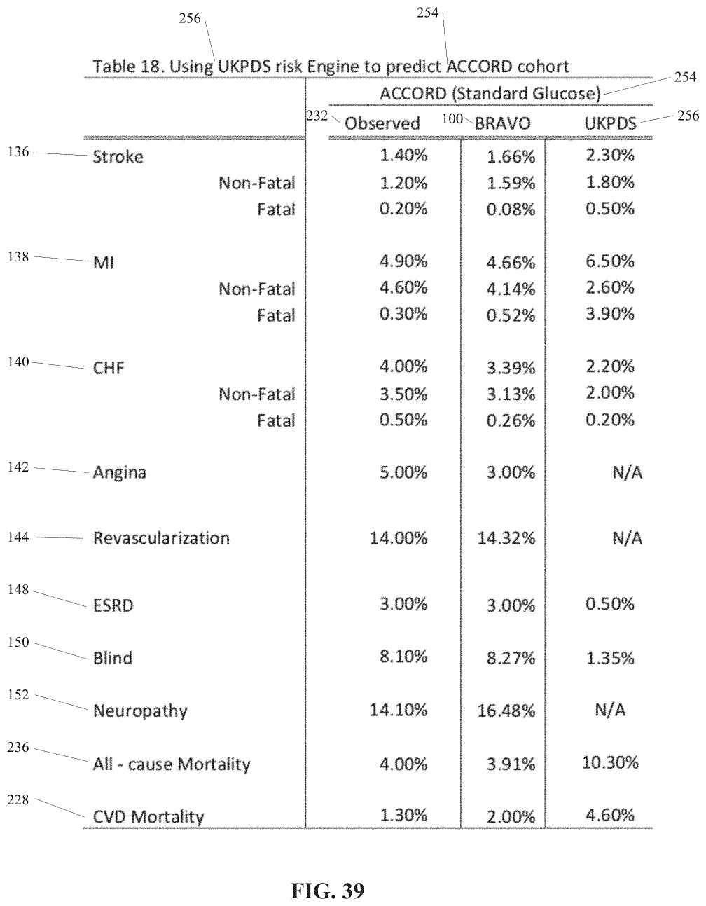

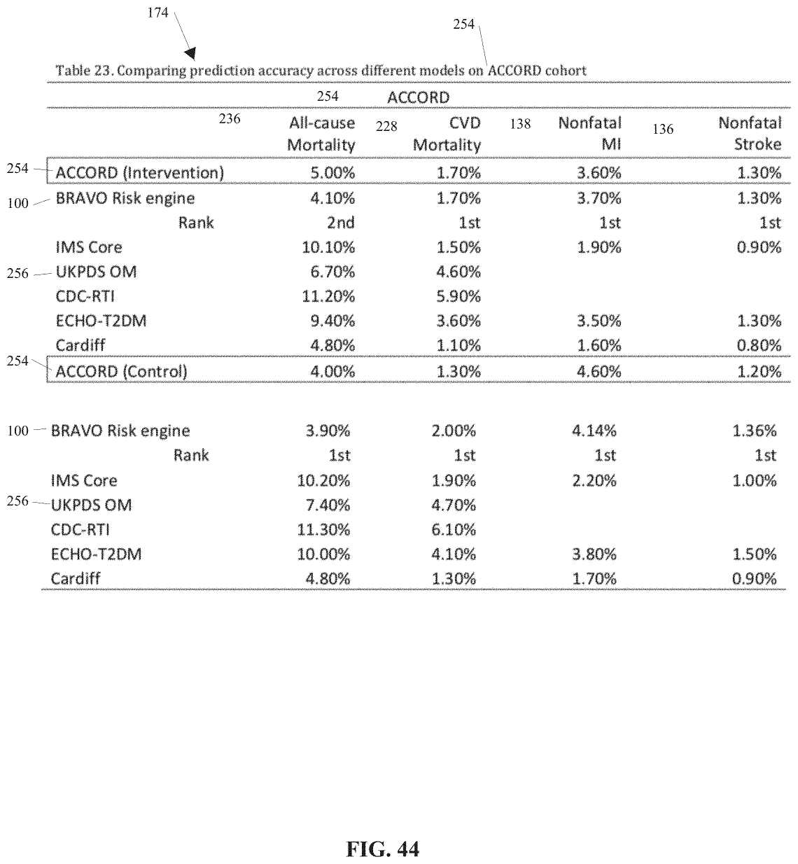

Referring to FIGS. 4, 13, 39 and 44, ACCORD 254 is one of the largest studies ever conducted in adults with type 2 diabetes who were at especially high risk of cardiovascular events 134, such as heart attacks 138, stroke 136, or death from cardiovascular disease 228. The multicenter clinical trial tested three potential strategies to lower the risk 192 of major cardiovascular events 134: intensive control of blood sugar, intensive control of blood pressure, and treatment of multiple blood lipids. The lipids targeted for intensive treatment were high density lipoprotein (HDL) cholesterol and triglycerides, in addition to standard therapy of lowering low density lipoprotein (LDL) cholesterol 132.

ACCORD 254 researchers from 77 medical centers in the United States and Canada studied 10,251 participants between the ages of 40 and 79 who had type 2 diabetes for an average of 10 years. When they joined the study, all participants were at especially high risk of cardiovascular events 134 because they had pre-existing cardiovascular disease 228, evidence of subclinical cardiovascular disease 228, or at least two cardiovascular disease 228 risk factors 202 in addition to diabetes.

All participants were enrolled in the ACCORD blood sugar treatment clinical trial 254 and maintained good control of blood sugar levels during the study. In addition, participants were enrolled in either the blood pressure trial or the lipid trial and were treated and followed for an average of about five years.

All the diabetes related events 110 were recorded during the study. Patients 122 in the intensive-therapy group attended monthly visits for the first 4 months and then every 2 months thereafter, with at least one interim phone call, with the aim of rapidly and safely reducing glycated hemoglobin 126 levels to below 6.0%. Additional visits were scheduled as needed to achieve glycemic 156, 158 goals, as described previously. Patients 122 in the standard-therapy group had glycemic-management 196 visits every 4 months.

The ACCORD trial 254 began in January 2001. The glycemia trial was terminated due to higher mortality 178 in the intensive compared with the standard glycemia treatment strategies. Study-delivered treatment for all ACCORD 254 participants was stopped on Jun. 30, 2009. All participants are continuing to be followed in a non-treatment observational study.

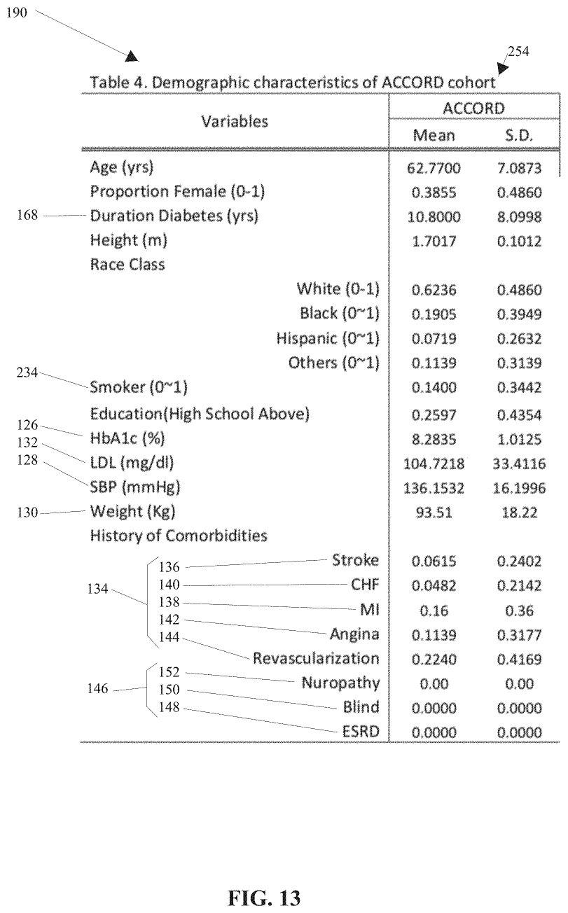

Detailed baseline characteristics 162 of the ACCORD cohort 254 are provided in FIG. 13.

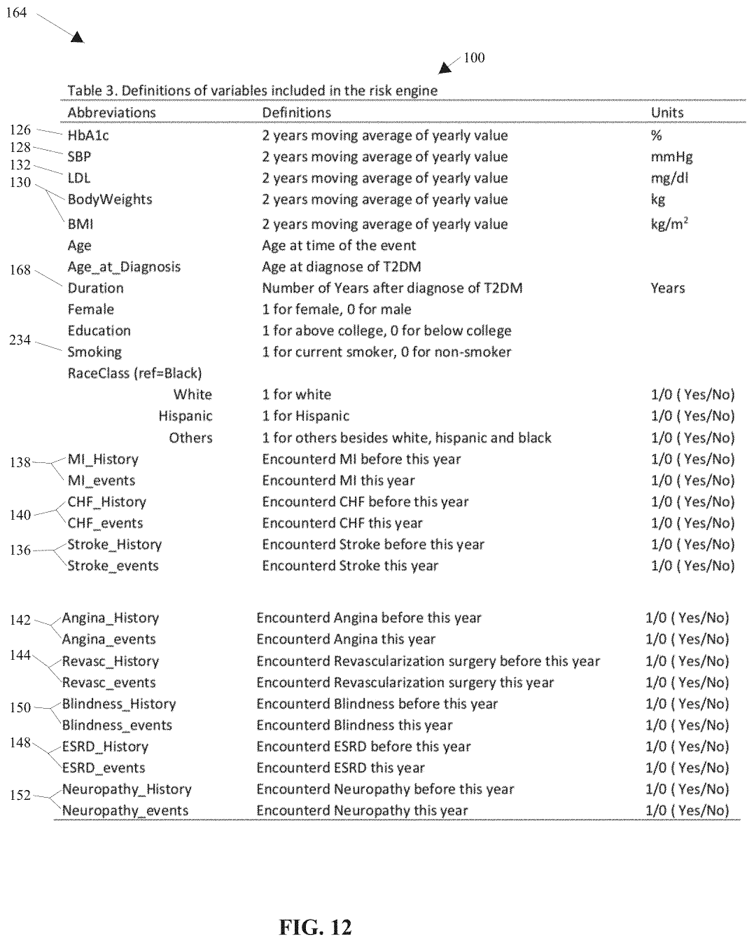

The Definition of all risk factors 202 initially included at the beginning of the variable selection process is provided in FIG. 12. Acknowledging the potential fluctuation at each measurement of biomedical factors 188 (i.e., HbA1c 126, SBP 128, LDL 132 and BMI 130), a moving-average technique is applied by calculating the average value of the previous 2 years to extrapolate the value for the current year. This approach improves the stability of the measurement on time-varying 170 factors 202 and has been previously applied to the UKPDS risk engine 256. An embodiment of the invention uses the value from last period to predict 174 the probability of current events 176, to account for potential bias caused by lag of measurement, as well as the reverse causal effect.

In the Events Module 108, eight exemplary risk equations 172 were fitted to predict Stroke 136, nonfatal MI 138, CHF 140, Angina 142, Revascularization Surgery 144, ESRD 148, Blindness 150, and neuropathy (measured by severe pressure sensation loss (SPSL) 152). The UKPDS risk engine 256 was used as the benchmark, combining current knowledge on other potential risk factors 202 as well as other potential functional forms 172 to improve the model fitting process. The clinical 186 definition for each event 110 were reported in the ACCORD trial 254 protocol.

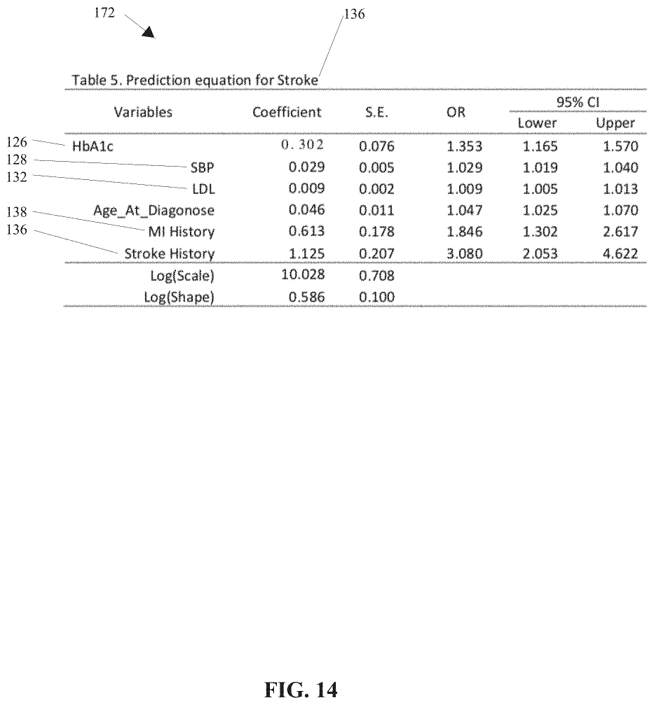

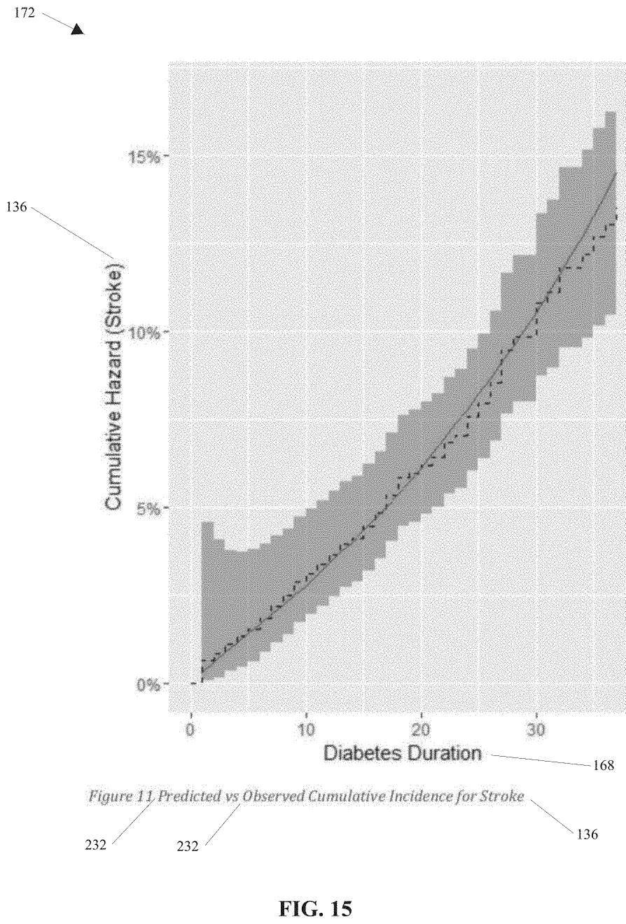

A Weibull survival regression 240 was fitted to predict 174 the hazard of stroke 136. Details regarding the parameter 164 estimates of the regression are provided in FIGS. 14-15. HbA1c 126, SBP 128, LDL 132, Age at diagnoses, MI 138 history and Stroke 136 history were included in the risk equation 172.

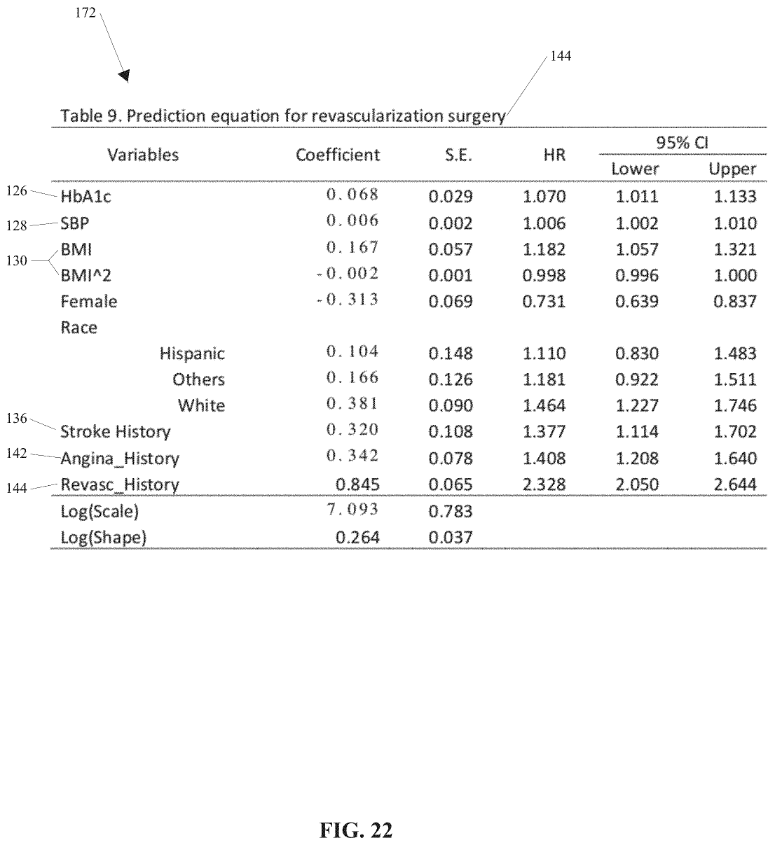

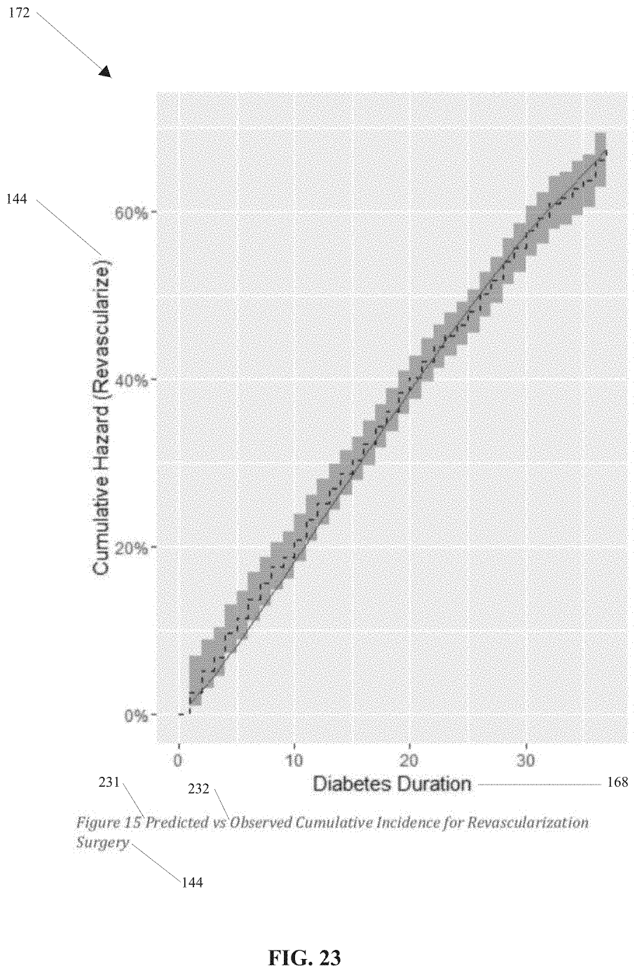

A Weibull survival regression 240 was fitted to predict 174 the hazard of CHF 140. Details regarding the parameter 164 estimates of the regression are provided in FIGS. 22-23. HbA1c 126, SBP 128, BMI 130, age at diagnose, severe hypoglycemia 156, education, MI 138 history, CHF 140 history and history of revascularization surgery 144 were included in the risk equation 172.

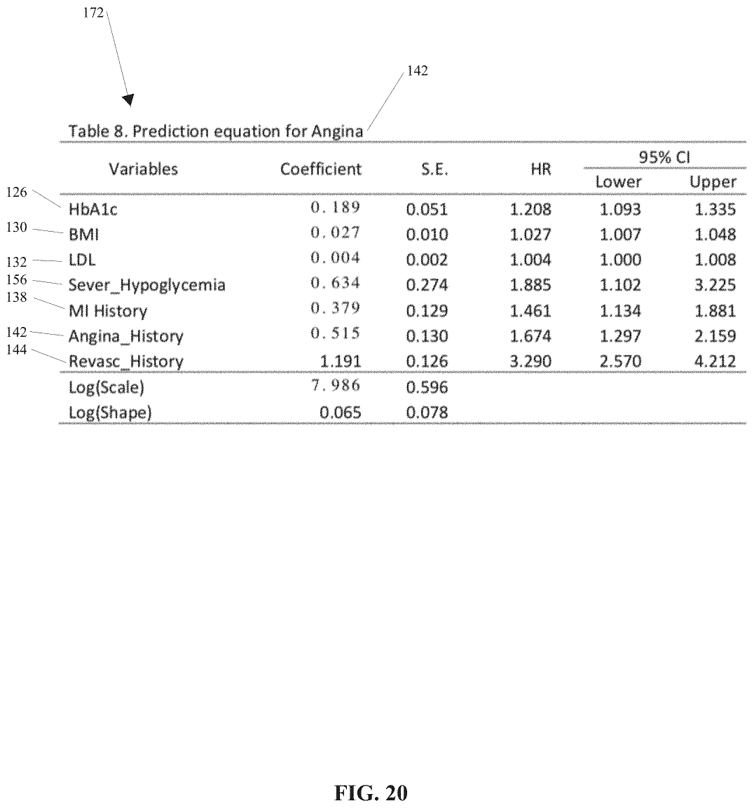

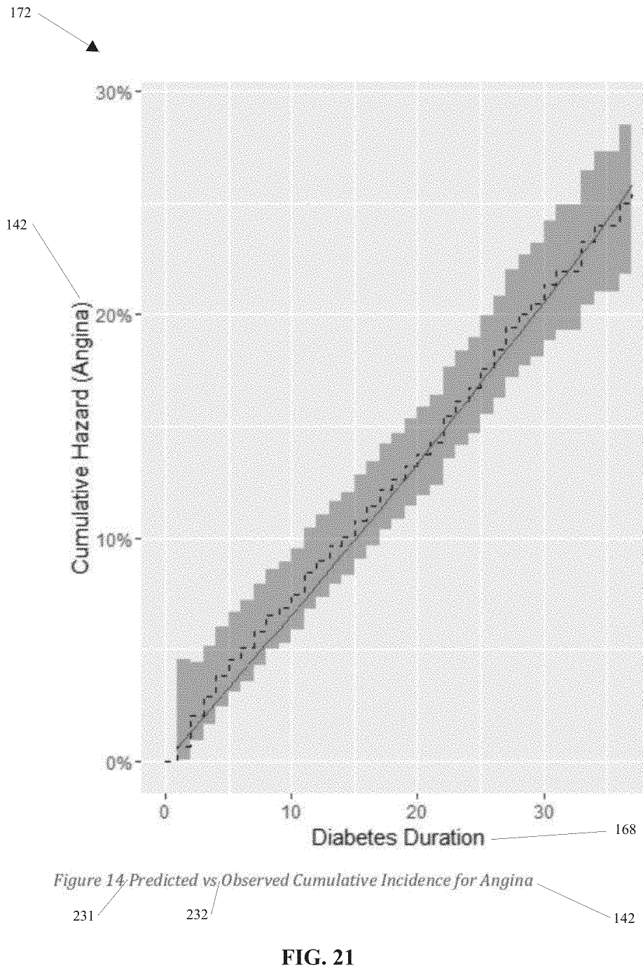

A Weibull survival regression 240 was fitted to predict 174 the hazard of angina 142. Details regarding the parameter 164 estimates of the regression are provided in FIGS. 20-21. HbA1c 126, SBP 128, female, race, stroke 136 history, angina 142 history and history of revascularization surgery 144 were included in the risk equation 172, as well as both first order BMI 130 and second order BMI 130.

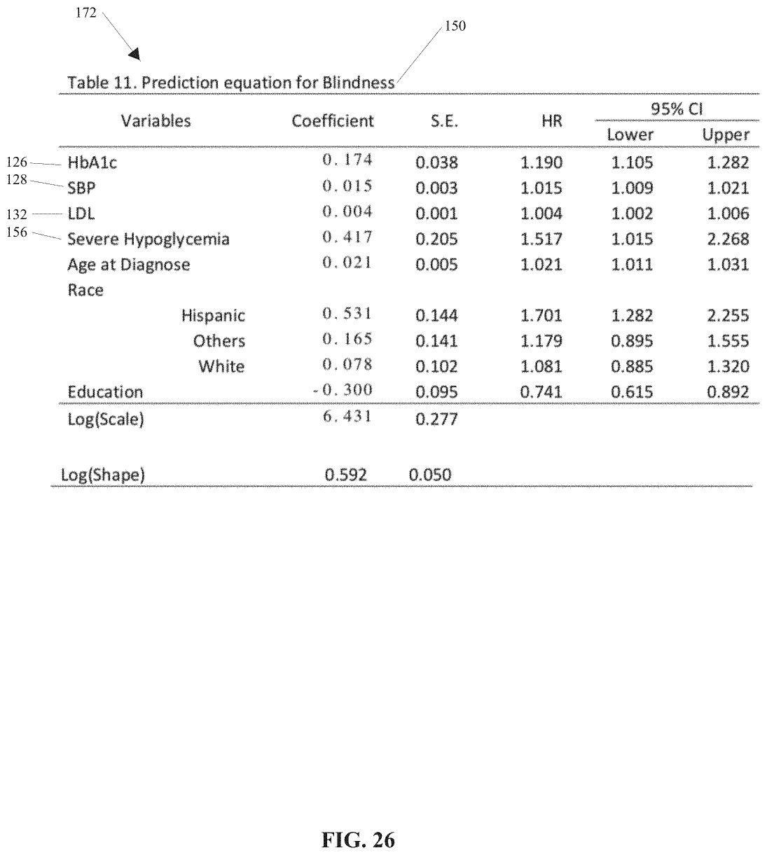

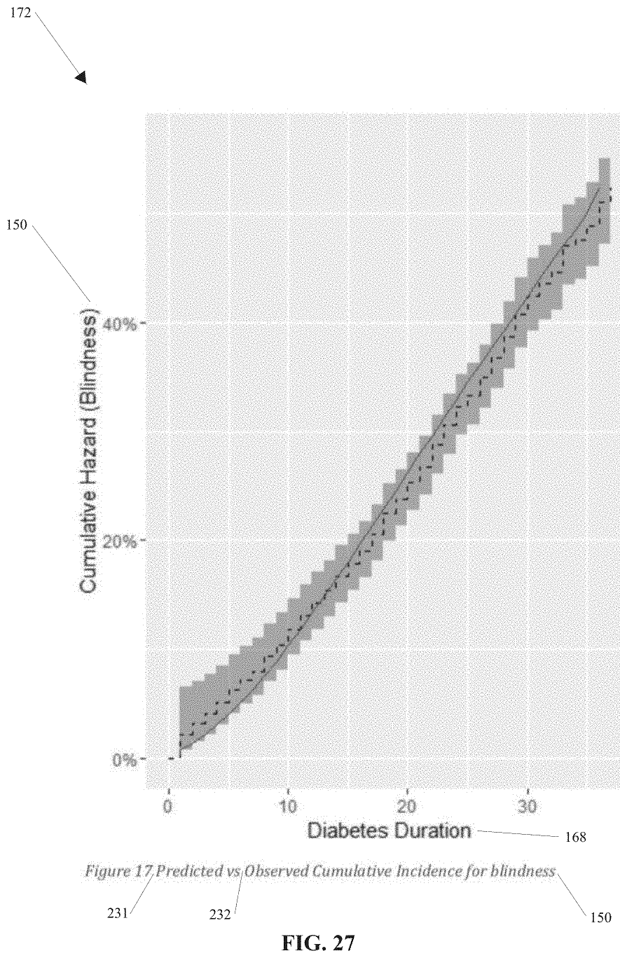

A Weibull survival regression 240 was fitted to predict 174 the hazard of blindness 150. Details regarding the parameter 164 estimates of the regression are provided in FIGS. 26-27.

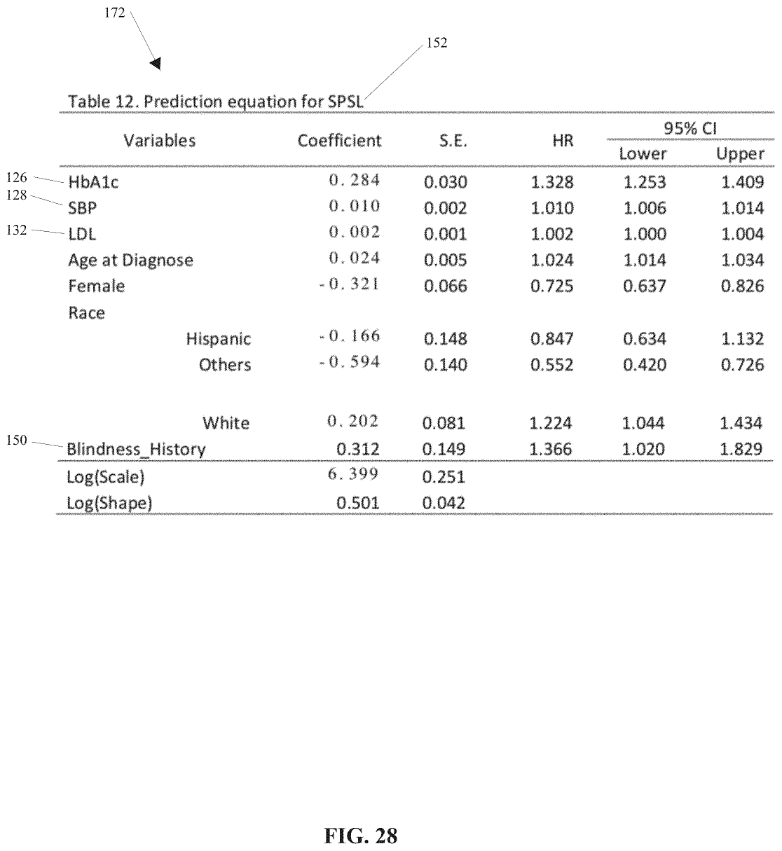

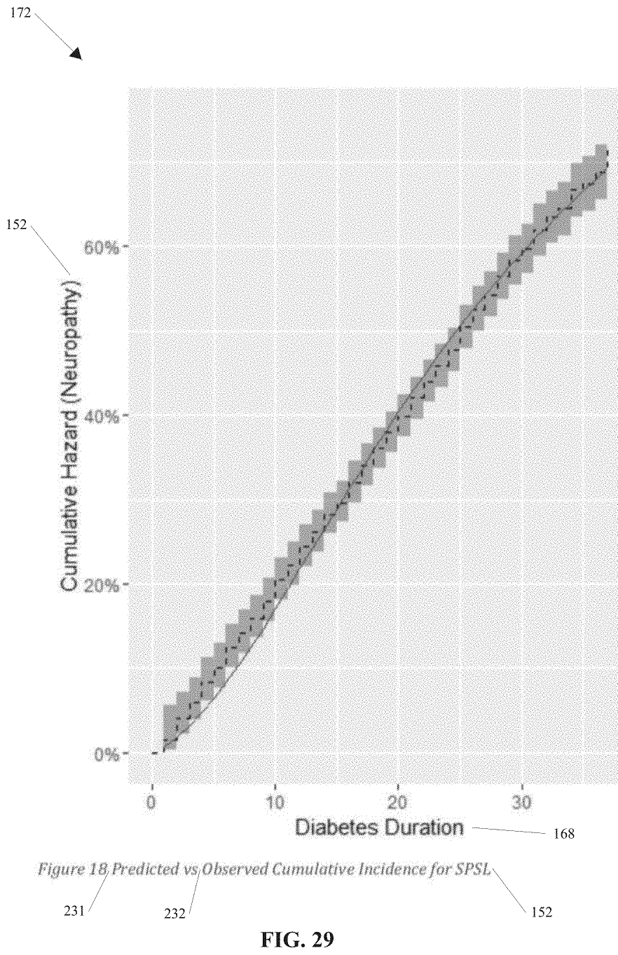

A Weibull survival regression 240 was fitted to predict 174 the hazard of neuropathy or SPSL 152. Details regarding the parameter 164 estimates of the regression are provided in FIGS. 28-29. HbA1c 126, SBP 128, LDL 132, age at diagnose, female, race, and retinopathy 150 history were included in the risk equation 172.

The risk factors module 114 of this embodiment tracked the progression 116 of HbA1c 126, SBP 128, LDL 132 and body weights 130 by fitting prediction equations 172 for each risk factor 202. In addition, as smoking 234 was identified as a risk factor 202 for several cardiovascular events 134, 146, a prediction equation 172 was fitted using previous and current smoking 234 status to predict the future smoking 234 status. At last, as one of the most important features of the BRAVO risk engine 100, a series of prediction models for predicting 174 both severe hypoglycemia 156 and symptomatic hypoglycemia 158 were fitted.

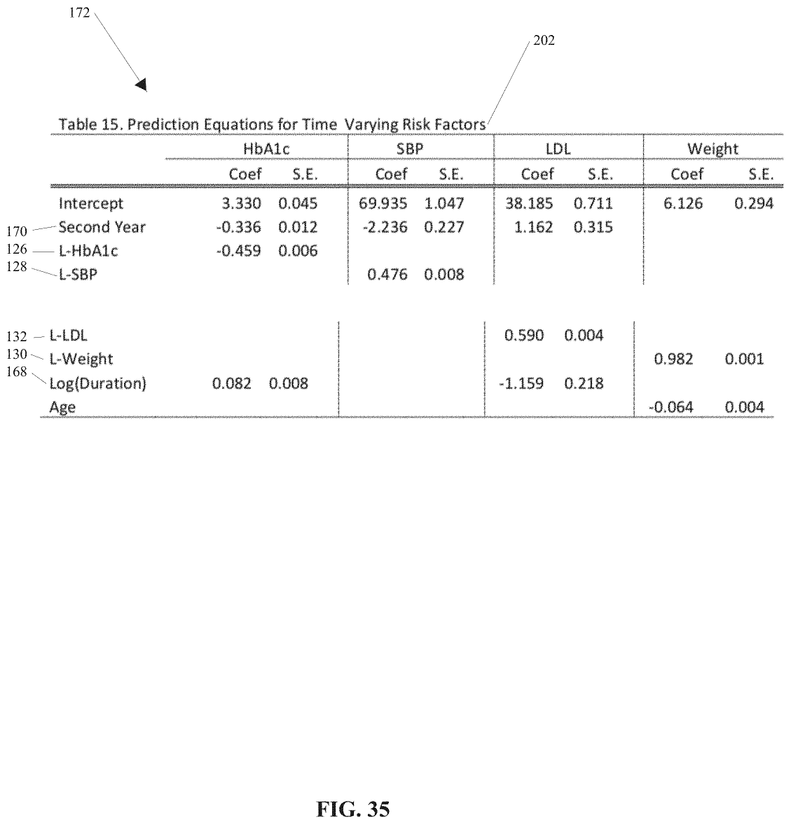

Parameter 164 estimates for OLS regressions 248 to predict 174 risk factors 202 are provided in FIG. 35. In general, the method is used to predict 174 the value of current year based on value from previous year. L-HbA1c 126 denotes the HbA1c 126 level at last year. For HbA1c 126, as evidence suggests an increasing trend over time 170, the method added the duration of diabetes 168 as a risk factor 202 to introduce the upward tendency over time 170. On the other hand, the body weights 130 for T2DMs tend to increase over time 170, but start to decline after around their 80s. The method included age as a covariates with negative coefficient to represent this declining trend.

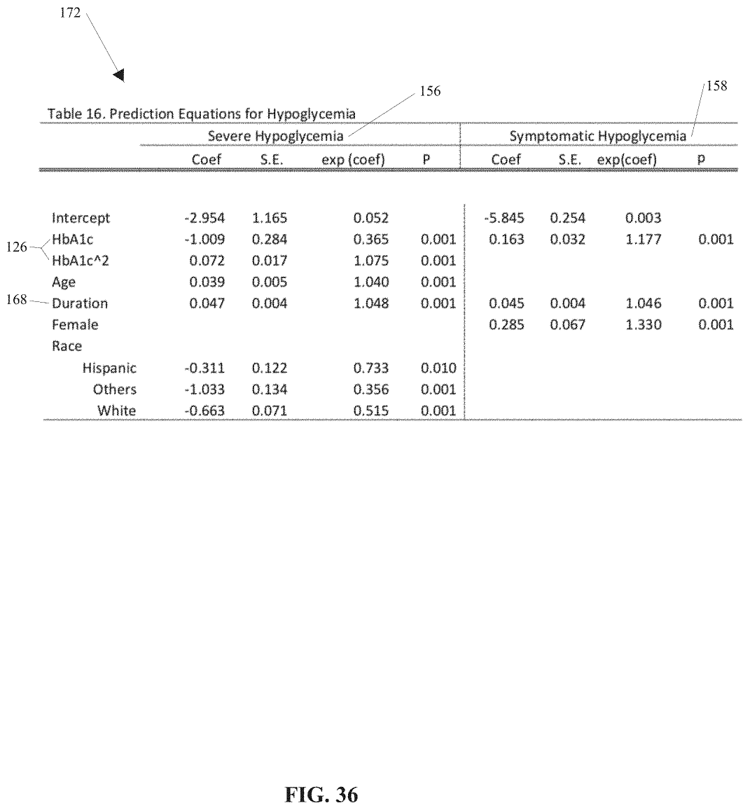

The BRAVO risk engine 100 uses two poisson regression models 246 to predict 174 the frequency of having severe hypoglycemia 156 and symptomatic hypoglycemia 158 in each cycle. The parameter 164 estimates are provided in FIG. 36.

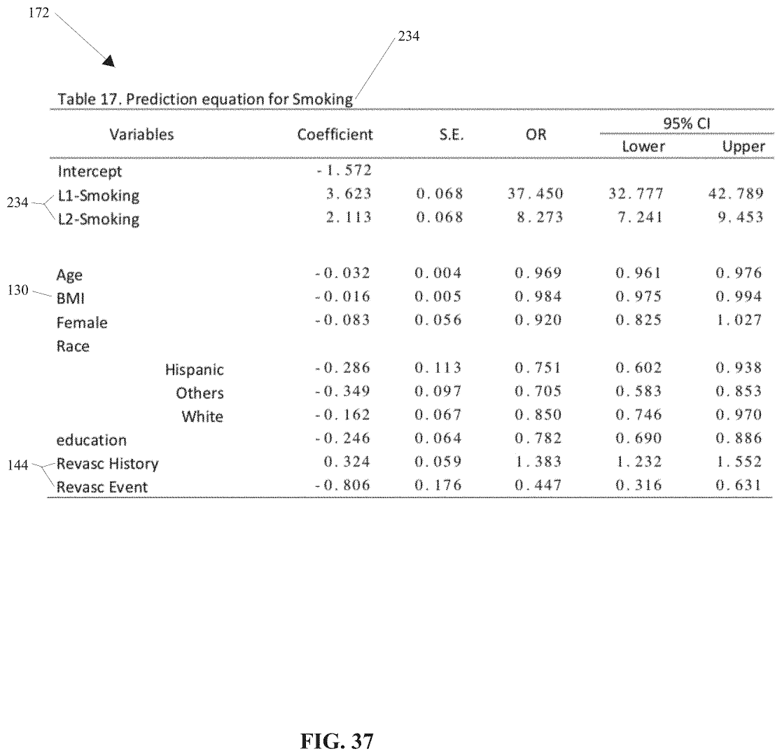

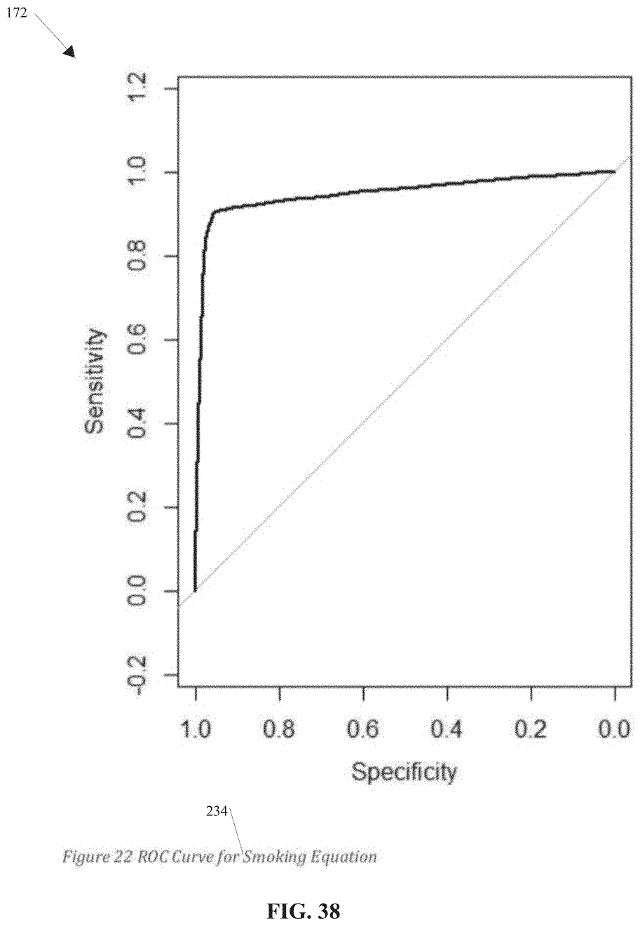

Since smoking 234 was identified as an important risk factor 202 to predict 174 all-cause mortality 236 and CVD 228 death, a logistic regression 244 was fitted to predict the likelihood of being a current smoker. Details regarding parameter 164 estimates are provided in FIGS. 37-38.

The mortality engine 118 of this embodiment used a proportional hazard model applied to predict 174 the all-cause mortality 236 rates during the lifetime of diabetes patients 122, to serve as the ceiling of the mortality risk 236. After that, a logistic regression 244 predicting 174 the probability of death 120 due to CVD 228 events was fitted among all patients 122 who encountered all-cause mortality 236. Weibull 240 distribution and Gompterz 242 distribution were both tested and compared to pick the baseline function that fitted the data best.

The parameters 164 for a fitted risk equation 172 is better described using a distribution of values, rather than a single value. In the BRAVO model 100, a boot-strapped method was applied by repeatedly resampling the population and re-running the risk equations 172 to generate the distributions of parameters 164 for each risk equation 172. Patient heterogeneity was handled using a patient-level microsimulation process, in which each patient 122 has different characteristics and simulation was carried out one person at a time.

Comparing to the Markov model approach, the BRAVO risk engine 100 may be based on a series of discrete inter-correlated risk equations 172. The discrete equations 172 approach accounts for the risk escalation 204 as diabetes progress 206 and interactions between complications 208. It also allows for adjusting a large number of demographic 190 and biological characteristics 188 and thus provides better estimation.

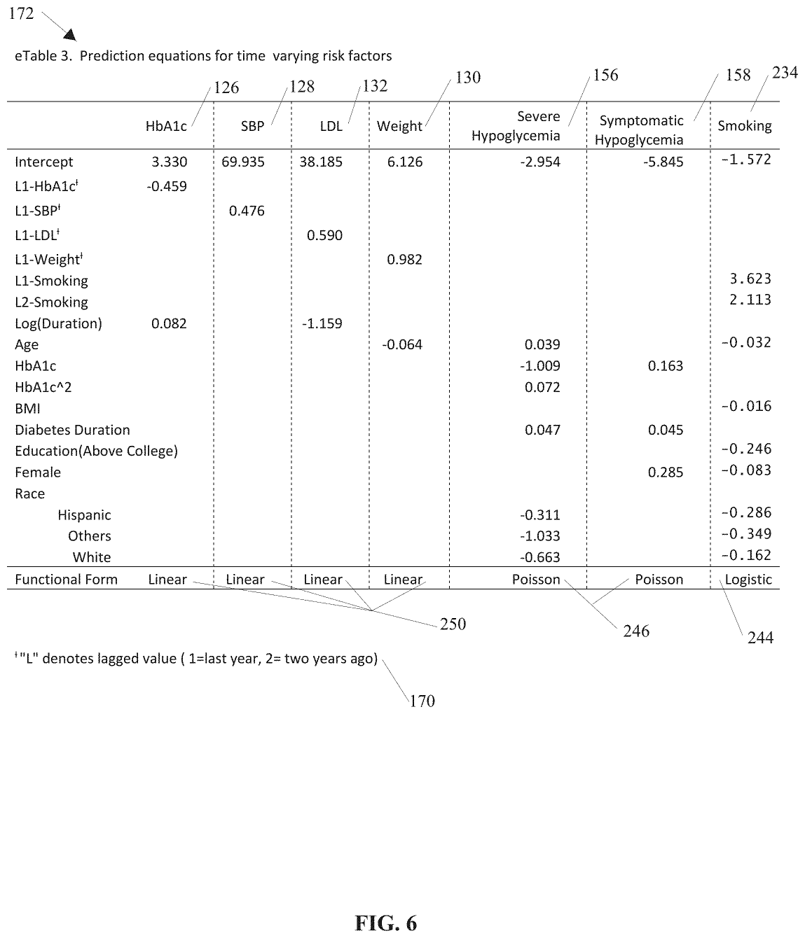

The BRAVO risk engine 100 tracks risk factors 202 over time 170. For example, as diabetes progresses 206, the function of β cells declines. This led to a constantly increased HbA1c 126 level across time 170. In addition, due to the feature of diabetes as well as a common side effect of diabetes treatment, the weight of T2DMs increased overtime. However, as people age, especially over 80 years old, their body weight 130 reduced over time 170. This lead to a reversed “U” shape of body weights 130 for diabetes patients over the course of diabetes 206. The risk engine 100 contains a series of equations 172 designed to represent the potential trend of change for each risk factor 202, as illustrated in FIG. 6.

The processors 102 and memories 104, or a subset thereof, can be configured to provide a means of corresponding to the methods (300 and 400) disclosed herein and various flowcharts and blocks of the Figures. Although not shown, the devices 220 may also include additional accessories and peripherals, such as security accessories, printers, and keyboards.

In one embodiment, as illustrated in FIGS. 3-8, the risk factor 202 is selected from the group consisting of glycosylated hemoglobin (HbA1c) 126, systolic blood pressure (SBP) 128, weight 130, and low-density lipoprotein cholesterol (LDL-C) 132. In such embodiment, each risk factor 202 [glycosylated hemoglobin (HbA1c) 126, systolic blood pressure (SBP) 128, weight 130, and low-density lipoprotein (LDL) 130] in the current cycle was predicted jointly by its value from the last cycle and other risk factors 202.

In another embodiment, as illustrated in FIGS. 3-8, the diabetes-related event 110 is a macrovascular event 134. Exemplary macrovascular events 134 include stroke 136, myocardial infarction 138, congestive heart failure 140, angina 142, and revascularization surgery 144.

In yet another embodiment, the diabetes-related event 110 is a microvascular event 146. Exemplary microvascular events 146 include end stage renal failure 148, blindness 150, and severe pressure sensation loss 152, as illustrated in FIGS. 3-8.

In one embodiment, as illustrated in FIGS. 3-8, the diabetes-related event 110 is an adverse event 154. Exemplary adverse events 154 include severe hypoglycemia 156 and symptomatic hypoglycemia 158.

In an exemplary embodiment of the events module 108, a series of risk equations 172 may be fitted to predict 174 diabetes-related macrovascular events 134 (stroke 136, MI 138, CHF 140, angina 142, and revascularization surgery 144), microvascular events 146 (ESRD 148, blindness 150, and SPSL 152), and adverse events 154 (severe hypoglycemia 156 and symptomatic hypoglycemia 158), for example, as illustrated in FIGS. 2-8.

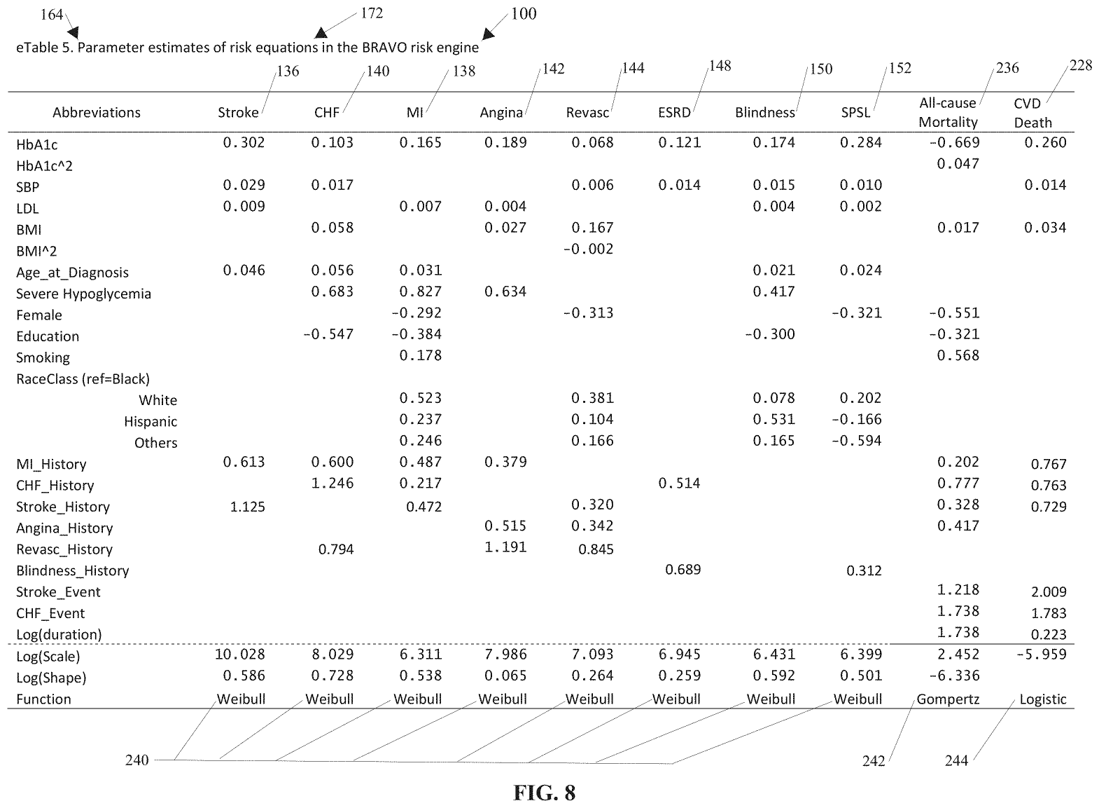

Details regarding the functional form of each risk equation 172 and model selection process, in accordance with embodiments disclosed herein, are provided in FIG. 2. Based on the type of each clinical outcome (i.e., time to event, binary, continuous and count), multiple functional forms were tested correspondingly. Referring to FIGS. 2 and 8, for time to event outcomes (i.e., macrovascular 134 and microvascular events 146 and all-cause mortality 236), Exponential, Weibull 240, Log-logistic 244, Log-normal, Gompertz 242 and Gamma distribution were tested. For binary outcomes (i.e. smoking 234 and CVD 228 death), logistic 244, probit and linear probability models 250 were tested, as shown in FIGS. 2, 6, and 8. For count outcomes (i.e., symptomatic 158 and severe hypoglycemia 156), Poisson 246 and negative binomial regressions were tested, as shown in FIG. 6. For continuous outcomes, as illustrated in FIG. 8, OLS 248 and Log-normal regression were tested. The ones with best prediction 174 accuracy were selected. The risk 176 of myocardial infarction (MI) 138, congestive heart failure (CHF) 140, stroke 136, angina 142, revascularization 144, blind 150, end stage renal disease (ESRD) 148 and severe pressure sensation loss (SPSL) 152 were modelled using Weibull survival model 240, as provided in FIG. 8. The risk of all-cause mortality 236 were modelled using Gompertz survival model 242. The risk of smoking 234 and cardiovascular (CVD) 228 death were modelled using logistic regression 244, illustrated in FIGS. 6 and 8. The value 164 of HbA1c 126, SBP 128, BMI 130 and LDL 132 were modelled using ordinary least squares (OLS) 248, as illustrated in FIG. 6. And the frequency of symptomatic hypoglycemia 158 and severe hypoglycemia 156 were modelled using poisson regression 246, as shown in FIG. 6. Functions of these models are shown in FIG. 2.

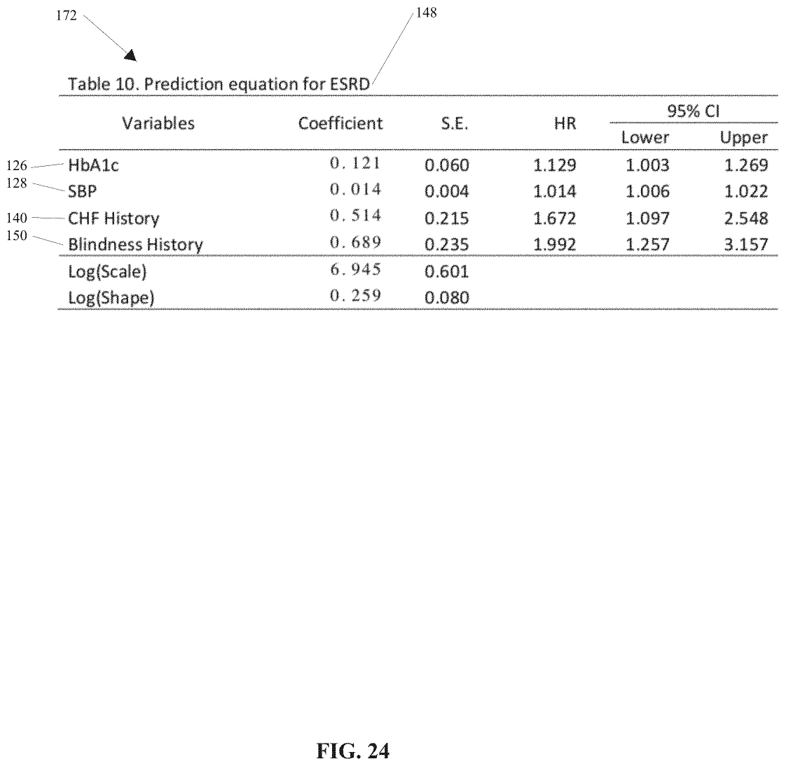

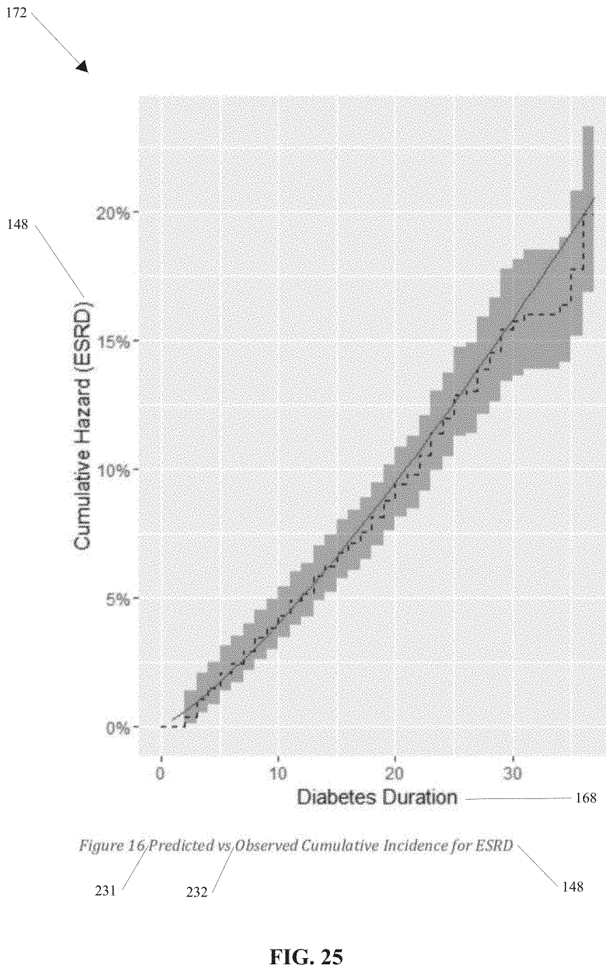

A Weibull survival regression 240 was fitted to predict 174 the hazard of ESRD 148. Details regarding the parameter 164 estimates of the regression are provided in FIGS. 24-25. HbA1c 126, SBP 128, CHF 140 history and history of blindness 150 were included in the risk equation 172.

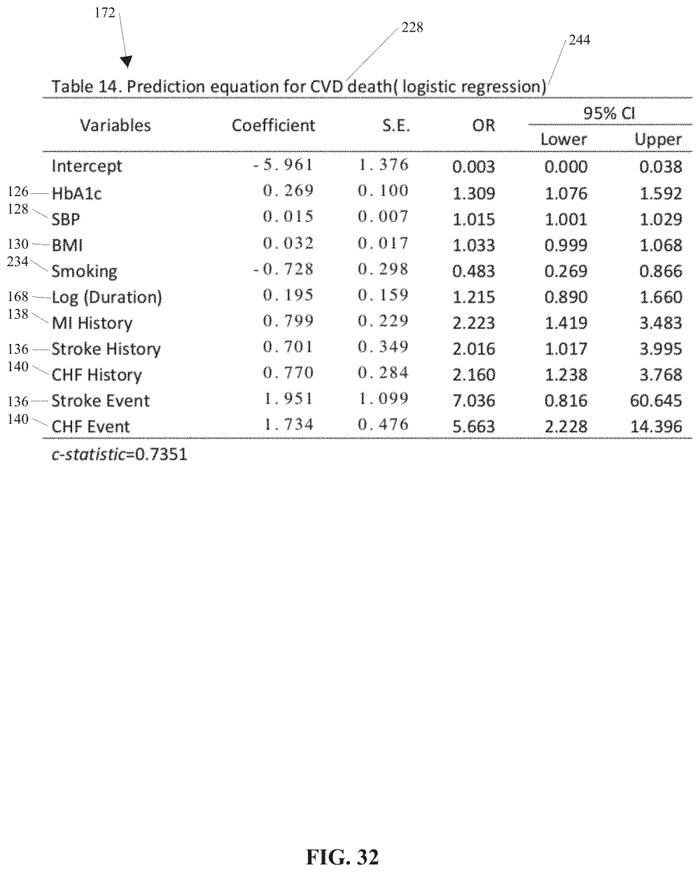

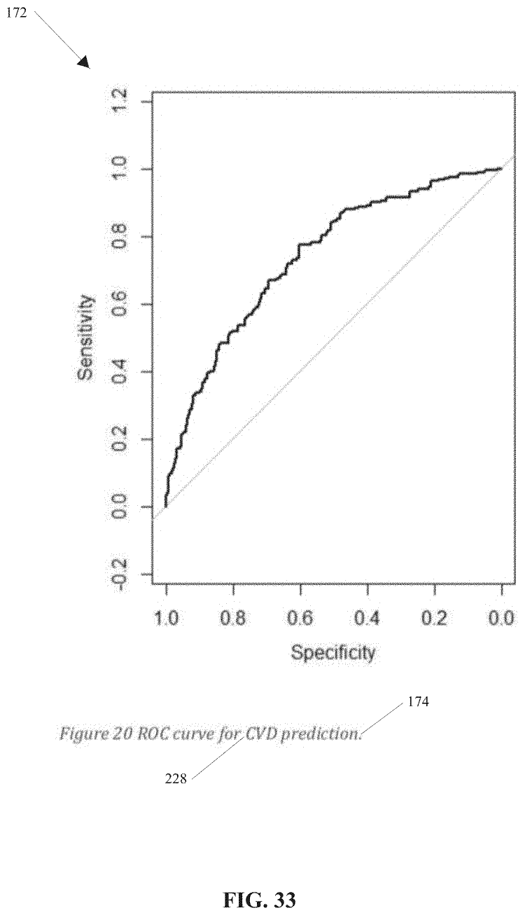

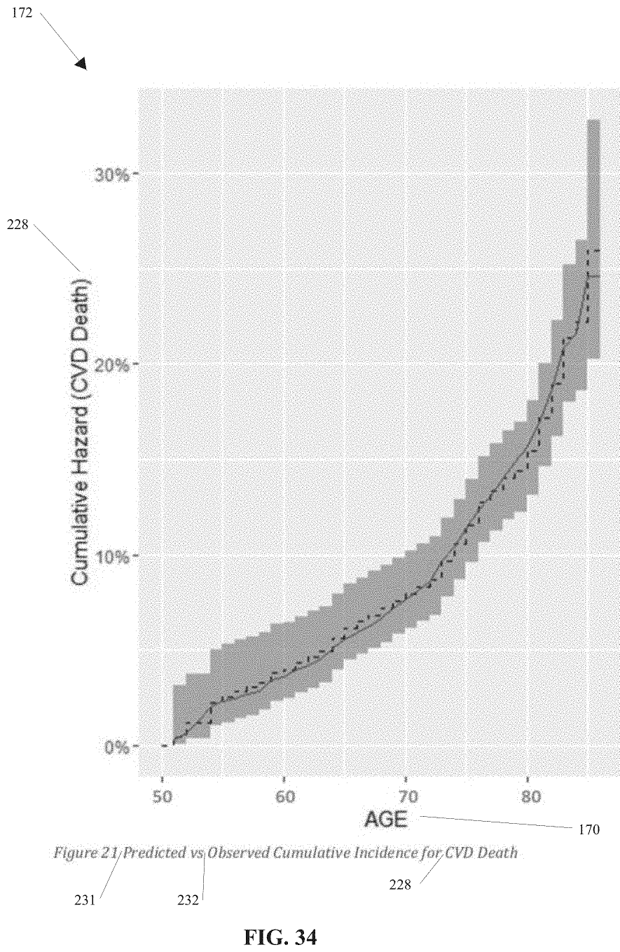

During the simulation process, if a person died in one cycle, the cause of that death would be decided based on equation (2) 172. A logistic regression 244 was fitted to predict 174 the likelihood for that death caused by CVD 228. Details regarding the parameter 164 estimates of the equation (2) 172 are provided in FIGS. 32-34.

Referring to FIG. 2, a Weibull survival model 240 was implemented in the risk equations 172. In the Weillbull survival model 240 “h” denotes the hazard rates of encountering the events 176 at time “t” (diabetes duration 168), or to be more specific, between time “t−1” and “t”. Shape and Scale are the two parameters 164 that co-specified the shape and size of the Weibull 240 distribution. “βX” is the linear form 250 of the regression model, in which “X” is a vector of included predictors 174 and “β” is a vector containing coefficients of each predictor 174. The Weibull survival model 240 was applied to predict 174 the incidence 192 of MI 138, CHF 140, Stroke 136, Angina 142, Revascularization 144, Blindness 150, ESRD 148 and neuropathy 152. The proportional hazard assumption was tested for each equation 172 through Schoenfeld residual plots for each of the predictors 174.

Referring to FIG. 2, a Gompertz survival model 242 was implemented in the risk equations 172. In the Gompertz survival model 242 “h” denotes the hazard rates of encountering the events 176 at time “t” 170, or to be more specific, between time “t−1” and “t”. Shape and Scale are the two parameters 164 that co-defined the shape and size of the Weibull 240 distribution. “βX” is the linear form 250 of the regression model, in which “X” is a vector of included predictors 174 and “β” is a vector containing coefficients of each predictor 174. The Gompertz survival model 242 was applied to predict 174 the all-cause mortality 236 rates.

Referring to FIG. 2, a Logistic regression model 244 was implemented in the risk equations 172. In the Logistic regression model 244 “Pr” denotes the probability 176 of encountering the events 134, 146, 154 at the current year. “βX” is the linear form 250 of the regression model, in which “X” is a vector of included predictors 174 and “β” is a vector containing coefficients of each predictor 174. Logistic regression model 244 was applied to predict 174 the smoking 234 status and CVD 228 death.

Referring to FIG. 2, a Poisson regression model 246 was implemented in the risk equations 172. In the Poisson regression model 246 “Count” denotes the expected frequency of the events 154 in that cycle. “βX” is the linear form 250 of the regression model, in which “X” is a vector of included predictors 174 and “β” is a vector containing coefficients of each predictor 174. Poisson regression 246 was applied to predict 174 the frequency of severe hypoglycemia 156 and symptomatic hypoglycemia 158.

Other datasets with relevant outcome measures may be used to either further refine prediction equations 172 or add supplementary risk equations 172 to the original BRAVO risk engine 100. Furthermore, referring to FIGS. 2 and 8, all the risk equations 172 were estimated separately and a microsimulation algorithm was used to combine them into one risk engine 100. Considering the mutually exclusive nature of some complications 210, this approach might still have competing risk bias. Moreover, the type of antidiabetic drugs were not included in the risk equations 172 because the underlying assumption of the BRAVO risk engine 100 is that all types of treatments, including lifestyle modification 214, impact the risk of events 176 only through key risk factors 202 (e.g., HbA1c 126, BMI 130). In one embodiment, the RECODe risk equation 252 may be used to directly explore the impact of different medications. Finally, ESRD 148 was found to be associated with a lower mortality rate 178 in the study, owing to a low sample size. Thus, as illustrated in FIGS. 24-25, ESRD 148 was excluded from the all-cause mortality 236 equation. However, ESRD 148 may be included upon receipt of the long-term follow-up data 180, 182 from the ACCORD trial 254.

In an exemplary embodiment of the mortality module 118, as illustrated in FIGS. 2-8, an equation 172 was fitted to predict 174 patient 122 death 178, and a second equation 172 was developed to explore the cause of death (i.e., cardiovascular disease (CVD) 228 or other death causes 230).

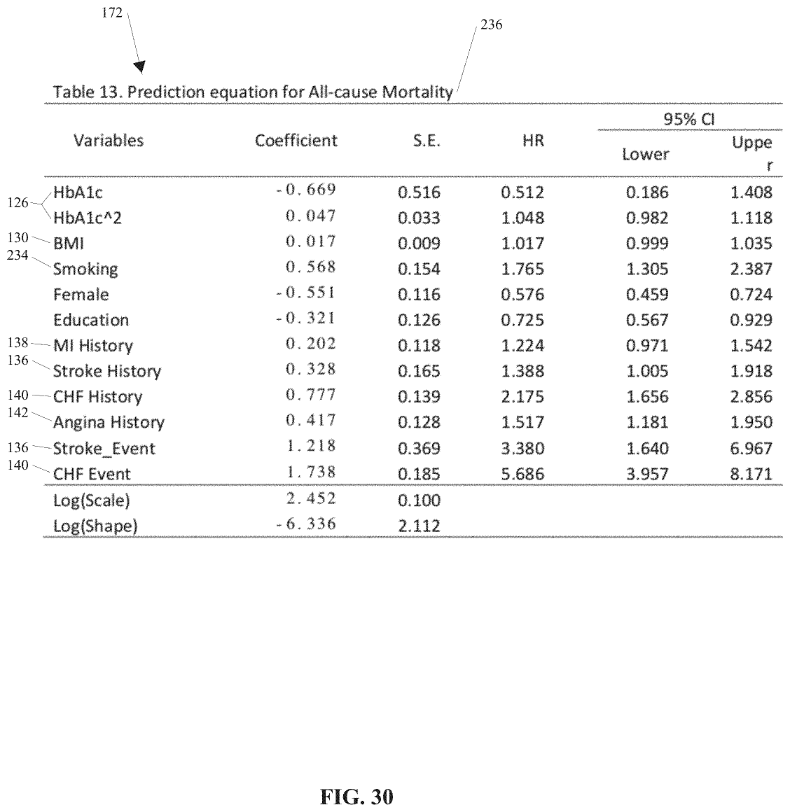

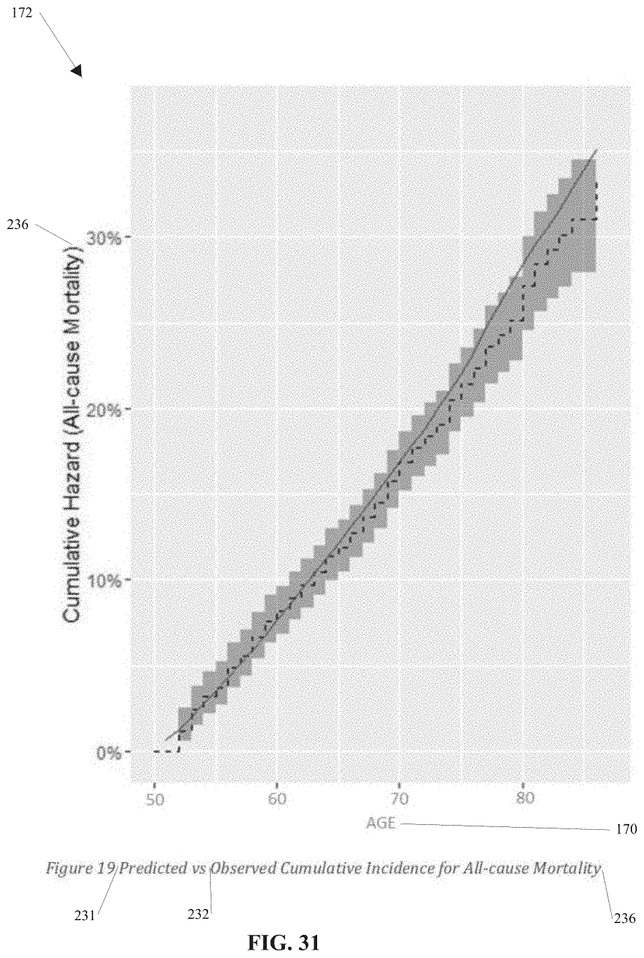

Referring to FIGS. 30-32, the mortality module 118 contains two equations 172: (1) an equation 172 predicting 174 the likelihood of all-cause mortality 236, and (2) an equation 172 predicting 174 the likelihood of CVD 228 death. The patients 122 would go through the all-cause mortality 236 equation 172 to decide whether he/she survived from this simulated cycle. If a patient 122 died during this cycle based on equation (1) 172, then the engine would calculate the likelihood of this death caused by CVD 228 using the equation (2) 172.

Embodiments of the invention include the Weibull survival regression 240 and Gompertz survival regression 242, which are fitted to predict 174 all-cause mortality 236. The Gompertz survival regression 242 provides a better fit according to the BRAVO risk engine 100 internal validation process. Compared to previous risk equations 172, this equation 172 was fitted using age as time index 170 instead of diabetes duration 168.

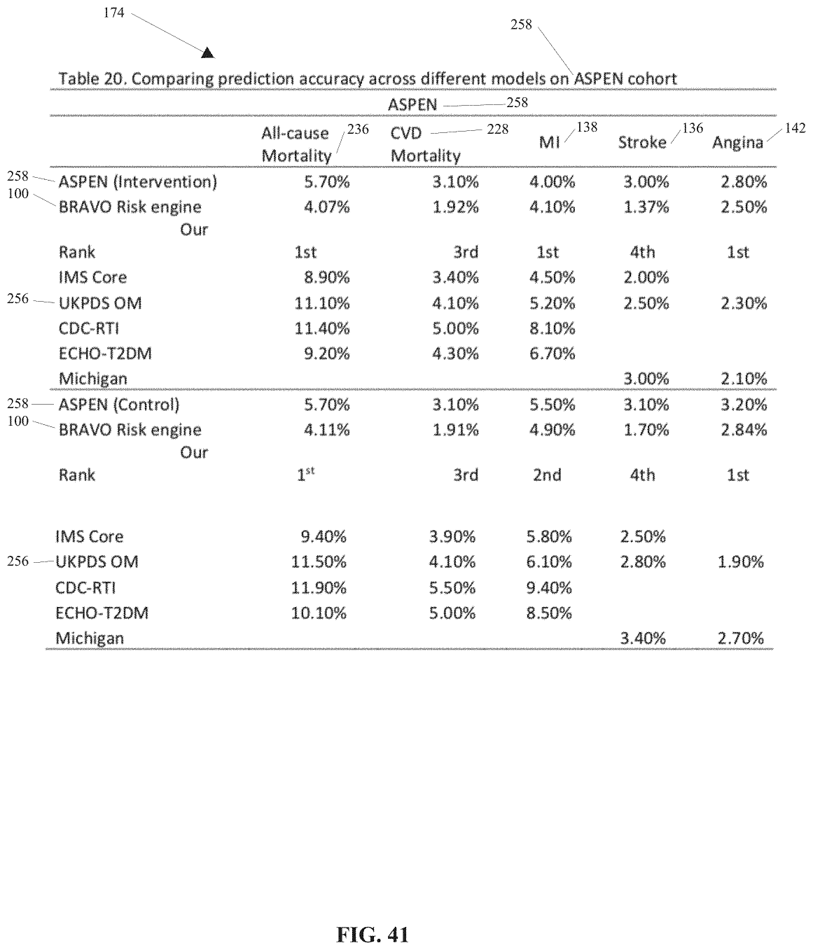

Details regarding the parameter 164 estimates of the equation (1) 172 are provided in FIG. 41. BMI 130, smoking 234, female, education, MI 138 history, CHF 140 history, angina 142 history, stroke 136 event, and CHF 140 event were included in the risk equation 172, as well as both first order and second order of HbA1c 126.

Referring to FIG. 12, the structure of the BRAVO risk engine simulation model 100 is illustrated. To reflect the chronic nature of diabetes, the model 100 follows T2DM patients from any specified time point to death or a user specified time horizon. “K” cohorts was generated from the user defined distribution of the population characteristics 166, and “I” patients 122 for each cohorts was then simulated. For each patient 122 in the same cohort, demographic characteristics 190 and risk factors 202 were assigned based on the assigned cohorts' parameter values 164. And then, based on that information, the first year macrovascular 134, microvascular 146, adverse events 154 and mortality for this patient 178 were predicted 174 through the BRAVO risk engine 100 risk equations 172. All of the events 134, 146, 154 were predicted 174 at a random order to account for events 134, 146, 154 interdependency. After that, the comorbidities will be updated for this patient 122 and their survival status 198 will be checked. If this patient 122 is identified to be dead during this year, then the simulation for this patient 122 was finished and the model 100 moved on to the next patient 122 in the same cohort. If the patient 122 is not dead, risk factors 202 (i.e. HbA1c 126, SBP 128, weights 130, Lipids 132) were updated based on the equations 172 implemented in the BRAVO risk engine 100 and the simulation 100 moved on to the next cycle, and all the diabetes-related events 176 were predicted 174 based on newly updated risk factors 202. This iteration was kept going until either the patient 122 is dead 178 or the simulation reached its time horizon 170. And after all the patients 122 in this cohort have been simulated, the model moved on to the next cohort until all the cohorts are simulated. The incidence for each diabetes related 110 comorbidity and mortality rates 120 were recorded on a yearly basis.

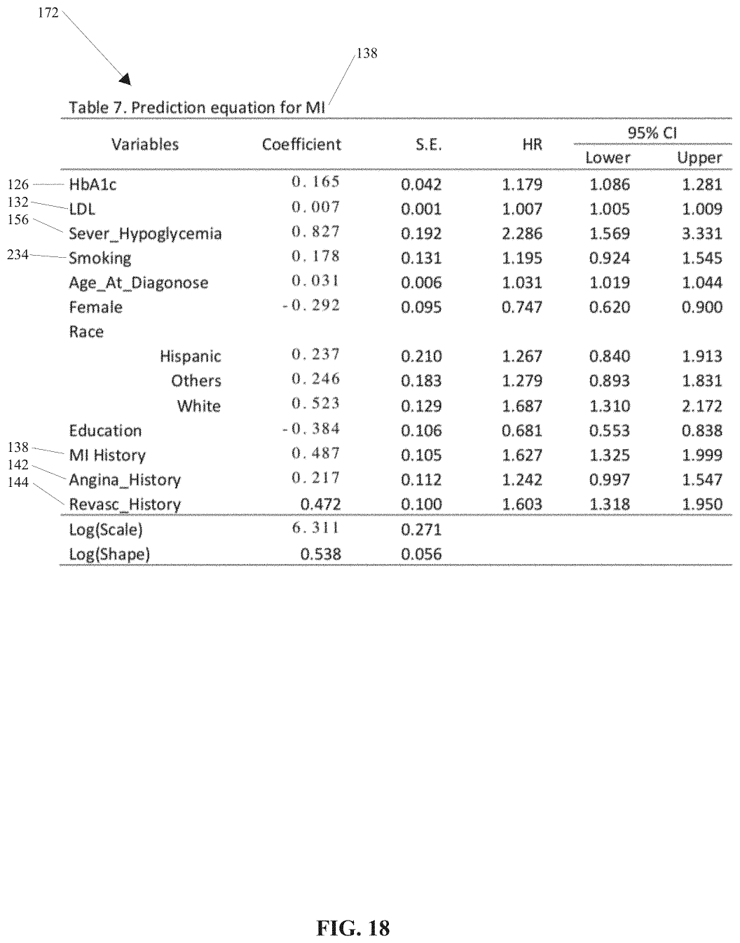

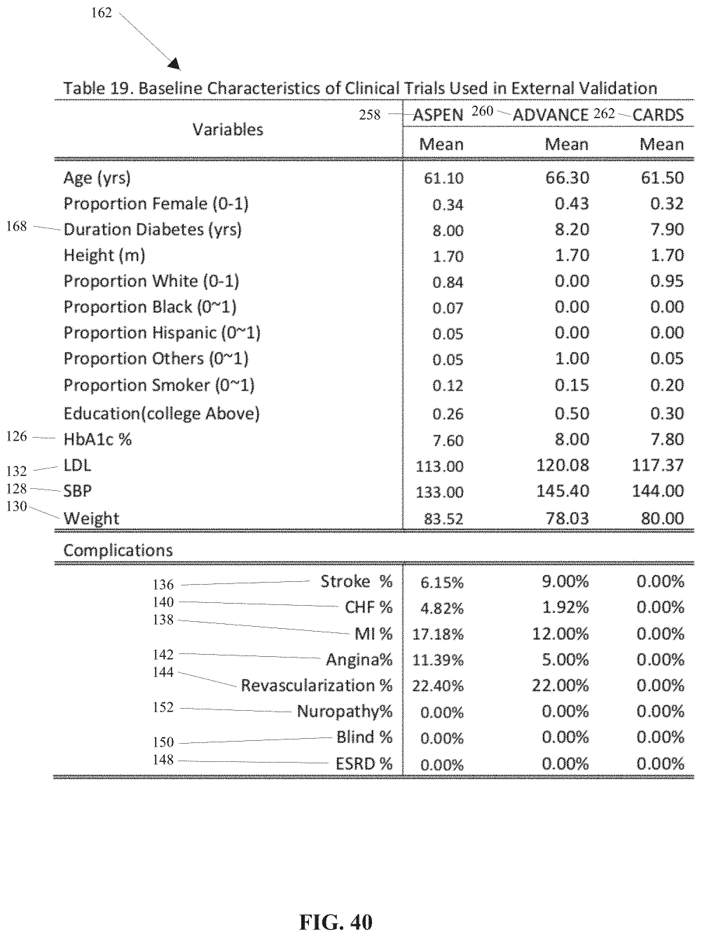

The BRAVO risk engine 100 with good internal and external validity, see FIG. 40, was developed to offer an alternative to the established UKPDS risk engine 256, an alternative that was based on the US population, to support decision making in US clinical practices. Health outcome predictions 174 from the BRAVO risk engine 100 were consistent with the previous findings of the ACCORD trial 254 on non-fatal MI 138, angina 142, and revascularization surgery 144, as illustrated in FIGS. 18-23. Although not statistically significant, a previous study has found that intensive glycemic control was associated with lower HRs of ESRD 148, blindness 150, and SPSL 152. The BRAVO risk engine 100 included HbA1c 126 as an important risk factor 202 for predicting 174 these microvascular events 146, as illustrated in FIGS. 24-29. More interestingly, even when including the extensive covariate list from the BRAVO risk engine 100, these associations between HbA1c 126 and microvascular events 146 were still statistically significant in the BRAVO risk equations 100.

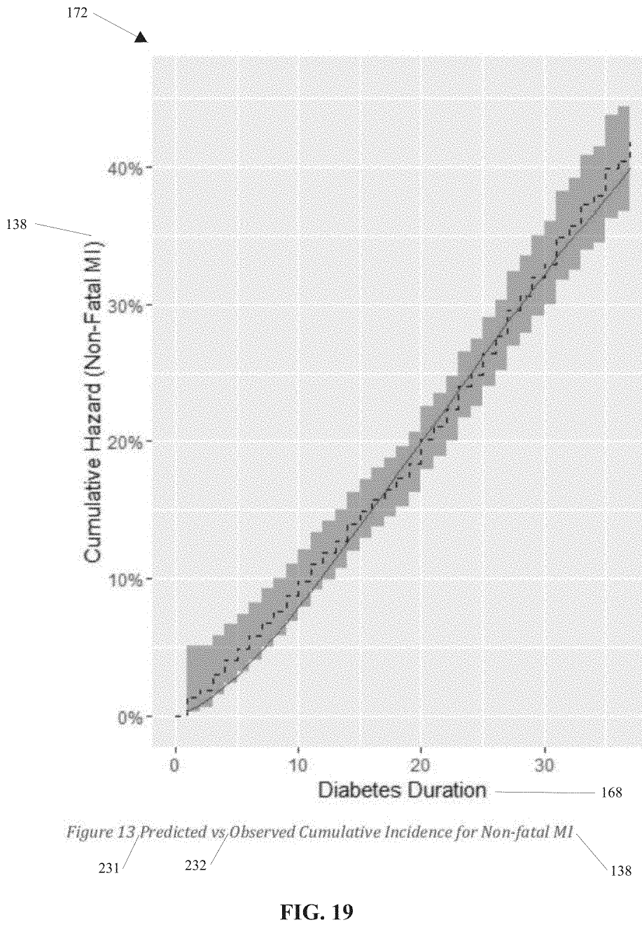

A Weibull survival regression 240 was fitted to predict 174 the hazard of non-fatal MI 138. Details regarding the parameter 164 estimates of the regression are provided in FIGS. 18-19. HbA1c 126, LDL 132, smoking 234, age at diagnose, female, severe hypoglycemia 156, race, education, MI 138 history, Angina 142 history, and history of revascularization surgery 144 were included in the risk equation 172.

The impact of hypoglycemia 156, 158 on diabetes outcomes and mortality 178 has been studied extensively in recent years. The occurrence of hypoglycemia 156, 158 was found to be associated with major macrovascular 134 and microvascular events 146, death 236, and other nonvascular outcomes. A previous study also found that in addition to the direct impact of hypoglycemia 156, 158 on vascular risk, the fear for hypoglycemia 156, 158 was also associated with an additional quality-adjusted life-year decrement. The risk engine 100 is the first to fully incorporate hypoglycemia's 156, 158 impact on disease course, as illustrated in FIG. 23.

Referring to FIG. 30, the engine 100 provides a critical predictive tool to evaluate new T2DM drugs, which usually have lower hypoglycemic 156, 158 incidents than the older class of antidiabetic drugs, such as sulfonylureas. One of the major limitations of T2DM models based on the UKPDS risk engine 256 is that they did not model hypoglycemia 156, 158 as a risk factor 202 for diabetes complications 210. To capture the impact of hypoglycemia 156, 158, the BRAVO risk engine 100 included severe hypoglycemia 156 as a risk factor 202 to predict 174 CHF 140, MI 138, angina 142, and blindness 150, as shown in FIG. 8. Encountering hypoglycemia 156, 158 was also found to be associated with higher CVD-related 228 mortality rates 178, as shown in FIGS. 30-34. This feature of the BRAVO risk engine 100 can directly capture the benefits of hypoglycemia 156, 158 prevention on cardiovascular 134, 146 outcomes and mortality 178 for future diabetes models, which is a substantial innovation, compared with previously developed diabetes-related risk engines 100.

The BRAVO risk engine 100 included severe hypoglycemia 156 in multiple risk equations 172. Encountering one more episode of severe hypoglycemia 156 in the current year was associated with increased risks for CHF 140 [hazard ratio (HR)=198%], MI 138 (HR=228.6%), angina 142 (HR=188.5%), and blindness 150 (HR=151.7%). Although not statistically significant, quadratic polynomials of HbA1c 126 levels were also found to be an important predictor 174 for predicting all-cause mortality 236 as indicated by Bier scores and c-statistics. An HbA1c 126 level of 7.12% was calculated to be associated with the lowest mortality risks 178. The equations 172 to model time-varying risk factors 202, including HbA1c 126, SBP 128, LDL 132, body weights 130, smoking status 234, and occurrence of severe hypoglycemia 156 and symptomatic hypoglycemia 158, are presented in FIG. 6.

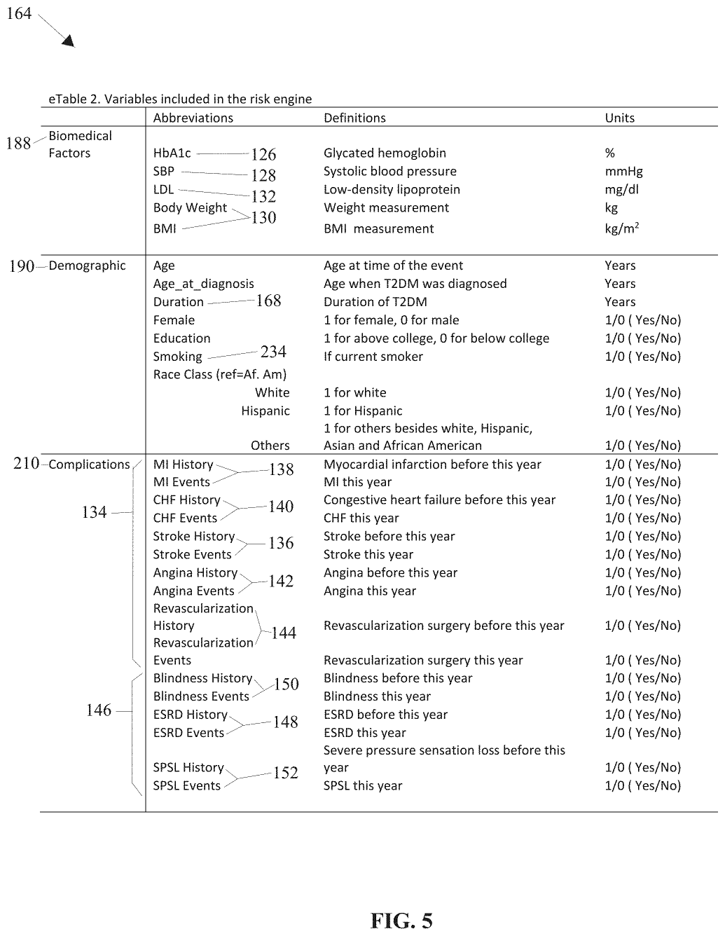

The existence of racial disparities in outcomes among a wide range of diabetic complications made it essential for a diabetes model to include race segmentation relevant to the target population for clinical intervention. Referring to FIG. 5, the BRAVO risk engine 100 categorizes race into Caucasian, African American, Hispanic, and Asian individuals in accordance with their representation in the US population. The BRAVO risk engine 100 predicted disparity patterns close to the finding from the Karter et al. study. Among all four race groups, being white was associated with the highest risk for MI 138 and nephropathy 238, while being African American was found to be a protecting factor with the lowest risk for MI 138, blindness 150, and the need for revascularization surgery 144.

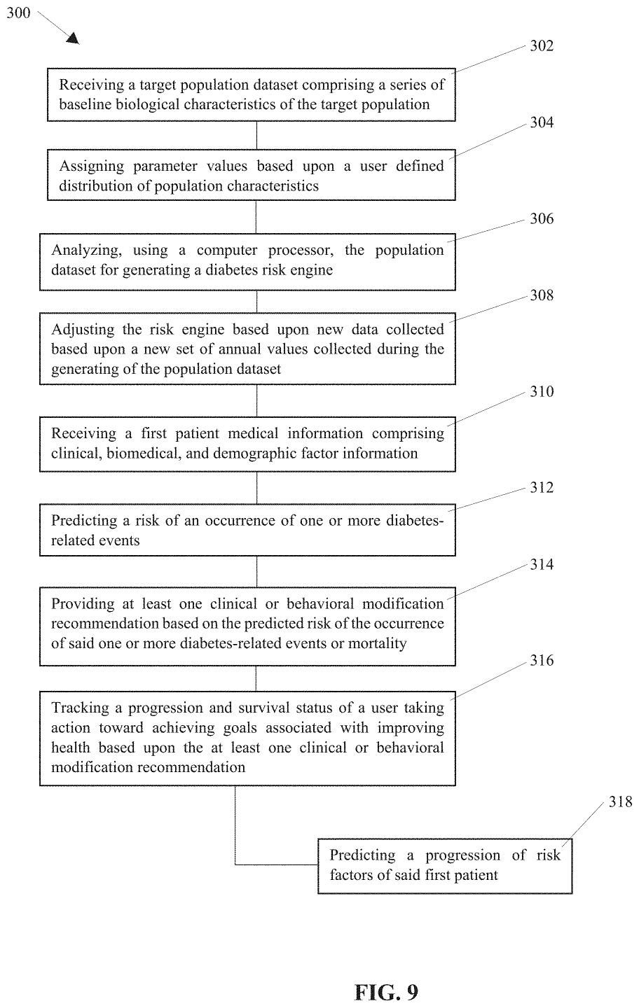

Therefore, in accordance with embodiments of the invention, there is provided a method 300 of predicting diabetes progression 206 and mortality 178. Referring to FIG. 9, the method 300 includes a first step of receiving 302 a target population dataset 160 having a series of baseline biological characteristics 162 of the target population 160. The method 300 has a second step of assigning 304 parameter values 164 based upon a user defined distribution of population characteristics 166. A third step of analyzing 306, using a computer processor 102, the population dataset 160 for generating a diabetes risk engine 100, wherein diabetes duration 168 is used as a time index 170. The diabetes risk engine 100 operates one or more inter-correlated risk equations 172 which can be used as a predictor 174 to determine a risk of a diabetes-related event 176 or mortality of a patient 178. A fourth step of adjusting 308 the risk engine 100 based upon new data collected 180 and based upon a new set of annual values collected 182 during the generating of the population dataset 160. The method 300 has a fifth step of receiving 310 a first patient medical information 184 including clinical 186, biomedical 188, and demographic 190 factor information. The method 300 includes a sixth step of predicting 312 a risk 192 of an occurrence of one or more diabetes-related events 110 with the diabetes risk engine 100 based upon the parameter values 164 by comparing the first patient medical information 184 to the population dataset 160. A seventh step of providing 314 at least one clinical or behavioral modification recommendation 196 based on the predicted risk 192 of the occurrence of the one or more diabetes-related events 110 or mortality 178. The eighth step tracks 316 a progression and survival status 198 of a user 200 taking action toward achieving goals associated with improving health based upon the at least one clinical or behavioral modification recommendation 196.

In one embodiment, the method 300 further includes a step of predicting 318 a progression of risk factors 202 of the first patient 184, as shown in FIG. 9.

In another embodiment, as shown in FIGS. 2 and 9, the inter-correlated risk equations 172 account for risk escalation 204 based upon diabetes progress 206 and interactions 208 between complications 210.

In yet another embodiment, the first patient medical information 184 includes risk factors 202 such as medication adherence 212, lifestyle modification 214, and therapy escalation 216, as shown in FIG. 9.

In one embodiment, as shown in FIG. 9, the diabetes-related event 110 is a macrovascular event 134. Exemplary macrovascular events 134 include stroke 136, myocardial infarction 138, congestive heart failure 140, angina 142, and revascularization surgery 144.

In another embodiment, the diabetes-related event 110 is a microvascular event 146. Exemplary microvascular events 146 include end stage renal failure 148, blindness 150, and severe pressure sensation loss 152, as shown in FIG. 9.

In yet another embodiment, as shown in FIG. 9, the diabetes-related event 110 is an adverse event 154. Exemplary adverse events 154 include severe hypoglycemia 156 and symptomatic hypoglycemia 158.

Referring to FIG. 4, a literature review was conducted to identify the initial list of baseline biological characteristics 162 and a backward selection process was conducted to remove those with no improvement for model fitting, in accordance with embodiments of the invention. Baseline biological characteristics 162 can be categorized into groups such as biomedical factors 188, demographic characteristics 190, and complications 186, for example. The definitions of these baseline biological characteristics 162 are provided in FIG. 4. The main modeling strategy utilized a left-censored, time-dependent, parametric proportional hazard model, in which diabetes duration 168 was used as the time index 170, instead of real time in the clinical trial. To smooth measurement fluctuation for biomarkers, a moving average technique was applied: as the models were developed based on annual cycles, all the parameter values 164 should be aggregated annually. Parameter values 164 for each year were aggregated by averaging all the measurements conducted within the previous 2 years. Parameter values 164 from the current year were used to predict 174 the probability of encountering an outcome event 134, 146 in the next year to account for potential bias caused by reverse causality. A person having the ordinary skill in the art will appreciate that these parameters 164 may be varied depending on application specifics. The history of events was also included in the initial list of baseline biological characteristics 162. Having had an event 134, 146, 154 at baseline or during the study periods before the current year would both be identified as a history of that event for the current year. A mixed-method algorithm including a cross-validation-based, backward model selection process, literature review, and consultation from endocrinologists was used to support the model fitting process. For binary outcomes, the c-statistic has been applied to measure the discrimination power of the model.

As illustrated in FIG. 4, race was identified as a significant risk factor 202 for predicting 174 MI 138, revascularization surgery 144, blindness 150, and nephropathy 238. Compared with African Americans, Caucasian, Hispanic, and other race/ethnicities were associated with 68.7% (95% CI 31.0-117.2), 26.7% (95% CI—16.0 to 91.3), and 27.9% (95% CI—10.7 to 83.1) higher risks for MI 138, respectively. In addition, Caucasian, Hispanic, and other race/ethnicities were associated with a 46.4% (95% CI 22.7-74.6), 11.0% (−17.0 to 48.3), and 18.1% (95% CI—7.8 to 51.1) higher likelihood of receiving revascularization surgery 144, compared with African Americans. Caucasian, Hispanic, and other race/ethnicities were correlated with a risk escalation 204 of 8.1% (95% CI—11.5 to 32.0), 70.1% (95% CI 28.2-125.5), and 17.9% (95% CI—10.5 to 55.5), respectively, compared with African Americans for blindness 150. Last, Hispanic individuals and others were associated with risk reductions of SPSL 152 for 15.3% (95% CI—13.2 to 36.6) and 44.8% (95% CI 27.4-58.0), respectively, while Caucasians were associated with a 22.4% (95% CI 4.4-43.4) higher risk for SPSL 152 compared with African Americans.

The BRAVO risk engine 100 analyzed both the discrimination power of the model and prediction 174 accuracy. Thus, both the c-statistic and Brier score were calculated to support the model selection process. For continuous outcomes [HbA1c 126, SBP 128, LDL 132, and body mass index (BMI) 130], the mean square prediction error was used to select the models. A ten-fold cross-validation framework was applied to adjust the c-statistic, Brier score, and mean square prediction error for possible over-fitting in low-dimension regressions. All risk factors 202 that improved model performance were included into the final model. For those risk factors 202 that did not have a significant impact on model performance, inclusion and exclusion were judged by clinical endocrinology knowledge and evidence found from the current literature. Risk factors 202 that were not statistically significant in the BRAVO 100 model selection processes, but that were supported as risk factors 202 by existing clinical evidence 186, were included in the risk equations 172.

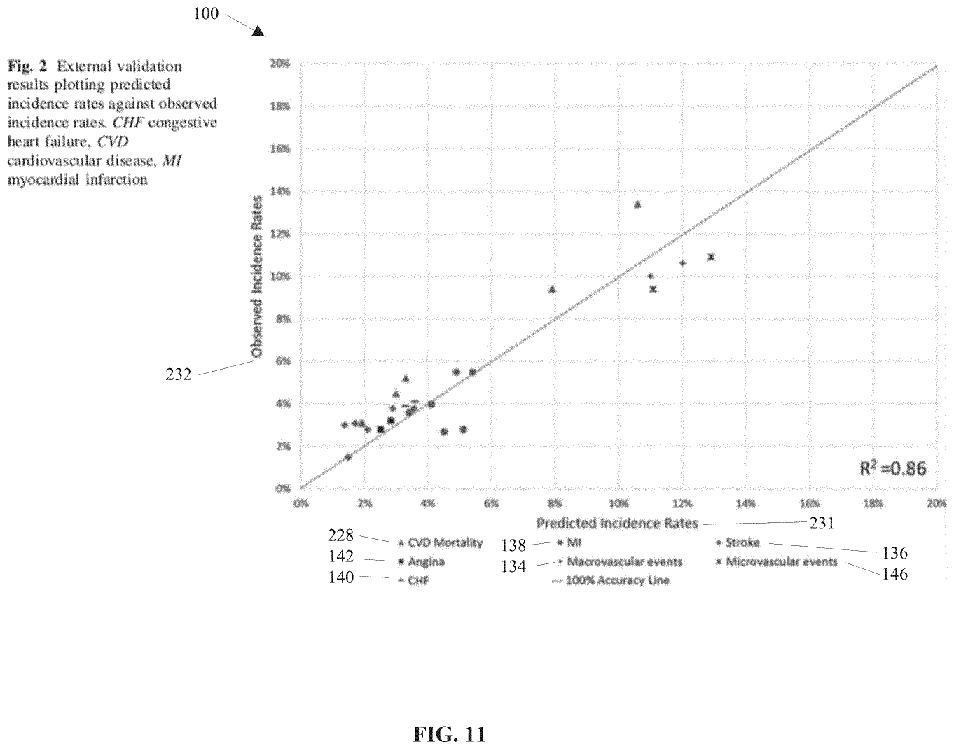

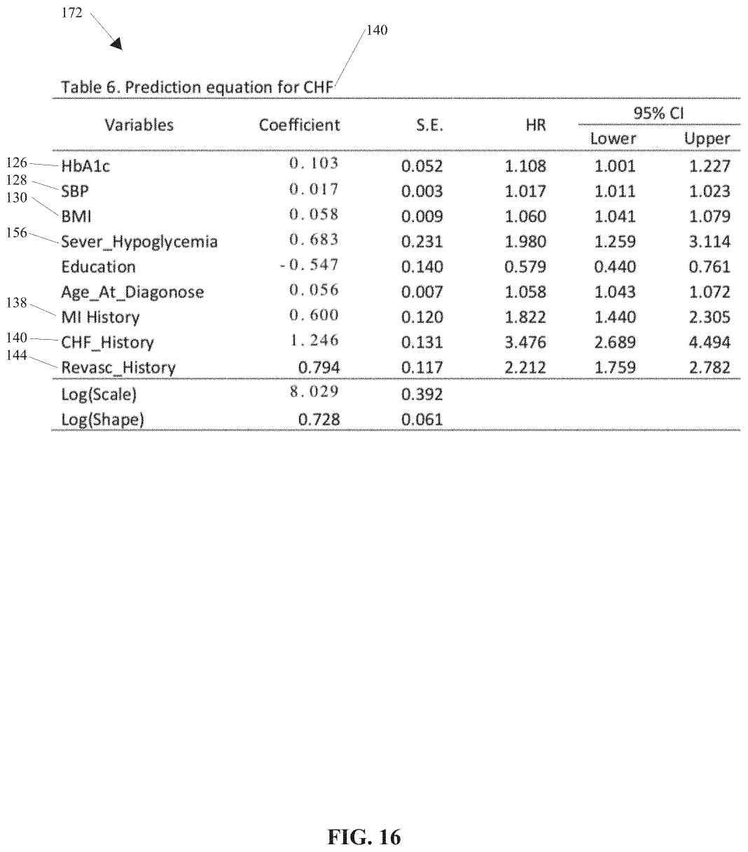

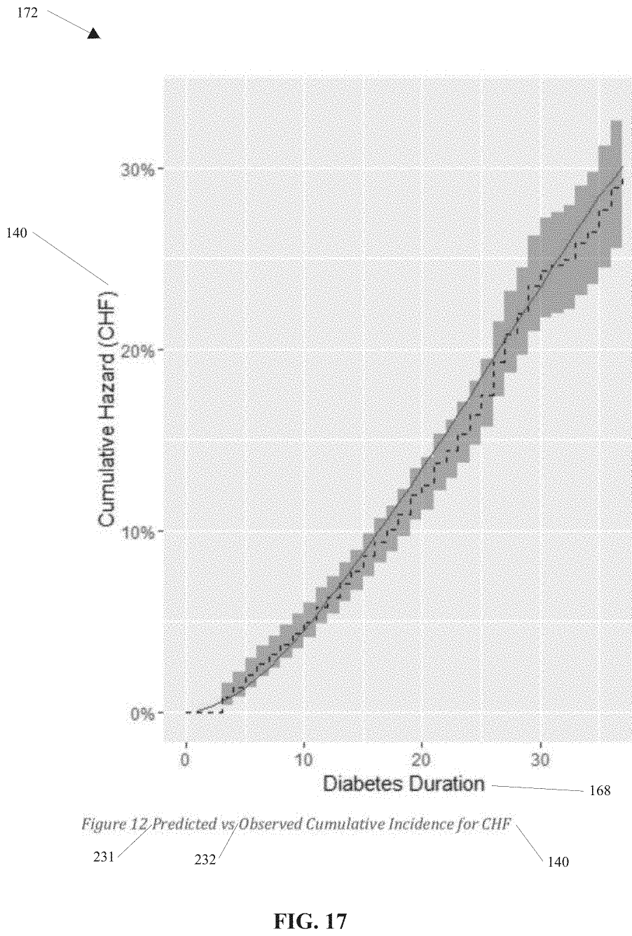

Referring to FIGS. 11-18, the internal validation process was conducted by plotting the predicted cumulative hazard against the Kaplan-Meier cumulative hazard for all outcome measures. The log-log 95% confidence interval (CI) for cumulative incidence rate across a diabetes duration 168 of 0-40 years in the ACCORD trial 254 was calculated using the left-truncated method, and the predicted curve was examined if it fell within the 95% CI of the Kaplan-Meier curve.

As the gold standard of internal validation, plotting the predicted cumulative hazard against Kaplan-Meiers cumulative hazard was applied to all the events prediction equations 172. For each timepoint from newly onset diabetes to 40 years after diagnoses were calculated using the log-log 95% confidence interval, and the predicted curve was examined if it falls within the 95% confidence interval of the Kaplan-Meiers Curve.

The predicted cumulative hazard was plotted over time, as shown in FIGS. 14-31. Plotted on the same chart is the Kaplan-Meier cumulative hazard for each age year. This process works as a supplement for the machine learning algorithm and is especially helpful on selecting the correct functional form (i.e. Weibull 240 or Gompertz survival models 242), shown in FIG. 2.

One of the important findings in the ACCORD trial 254 was a higher mortality rate 120 in the intensive glycemic control group (HbA1c \ 6%) compared with the standard glycemic control group (HbA1c 7.0-7.9%). The association between HbA1c 126 and mortality rate 178 was found to be ‘U’ shaped in the standard control group, with an optimal HbA1c 126 level between 7.0% and 7.5%. As illustrated in FIG. 6, the BRAVO risk engine 100 included a second-degree polynomial in the HbA1c 126 level that fits the data better than a linear relationship between the HbA1c 126 level and all-cause mortality 236. The BRAVO risk engine 100 estimated the optimal glycemic control level for the ACCORD 254 population was 7.12%, and any deviation from this point was associated with an increased risk of mortality 120.

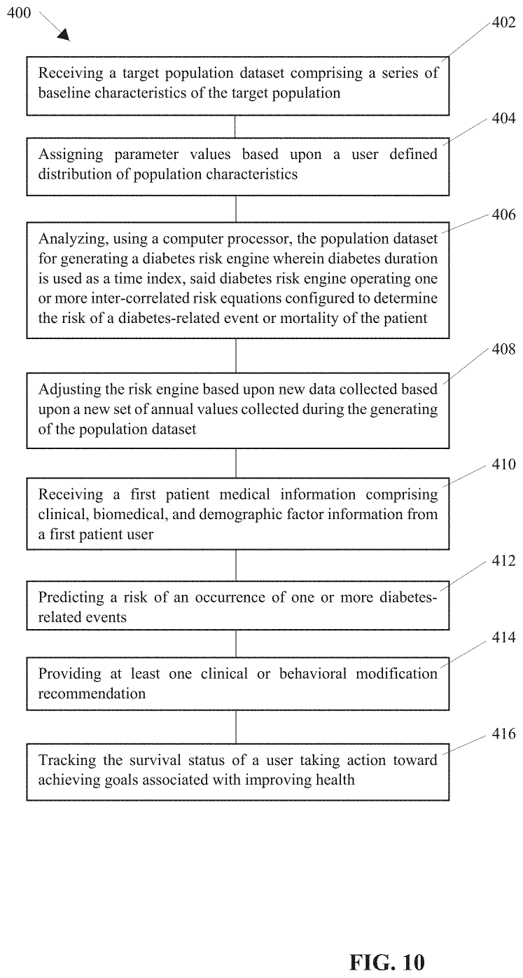

In accordance with embodiments of the invention, there is provided a method 400 of predicting an occurrence of one or more diabetes related events 110. Referring to FIG. 10, the method 400 has a first step of receiving 402 a target population dataset 160 comprising a series of baseline characteristics 162 of the target population 160. A second step of assigning 404 parameter values 164 based upon a user defined distribution of population characteristics 166. The method 400 includes a third step of analyzing 406, using a computer processor 102, the population dataset 160 for generating a diabetes risk engine 100 wherein diabetes duration 168 is used as a time index 170. In the third step 406, the diabetes risk engine 100 operates one or more inter-correlated risk equations 172 configured to determine the risk of a diabetes-related event 176 or mortality of a patient 178. A fourth step of adjusting 408 the risk engine 100 based upon new data collected 180 based upon a new set of annual values collected 182 during the generating of the population dataset 160. The method 400 includes a fifth step of receiving 410 a first patient medical information 184 such as clinical 186, biomedical 188, and demographic 190 factor information from a first patient user 194. A sixth step predicts 412 a risk 192 of an occurrence of one or more diabetes-related events 110 with the diabetes risk engine 100 based upon the parameter values 164 by comparing the first patient medical information 184 to the population dataset 160 of the diabetes risk engine 100. A seventh step provides 414 at least one clinical or behavioral modification recommendation 196 based on the risk 192 of the occurrence of said one or more diabetes-related events 194. An eighth step tracks 416 the survival status 198 of a user 200 taking action toward achieving goals associated with improving health based upon the at least one clinical or behavioral modification recommendation 196.

Referring to FIGS. 4-8, a total of 28 exemplary endpoints were predicted 174 using the BRAVO risk engine 100 through a discrete-time event microsimulation process under the corresponding time horizon of each trial. The baseline characteristics 162 of each trial have been reported and applied directly as the characteristic of simulation samples. Normal distribution was assumed for all input variables, as shown in FIG. 5, and the standard error of each variable was extracted from the corresponding literature. The values of key risk factors 202, including HbA1c 126, LDL 132, SBP 128, and BMI 130 in each validation trial were assumed to reach the corresponding treatment target 196 at the first year and remained constant in the following years. Then, 10,000 simulation runs were used to reach convergence in outcomes. An ordinary least-square model 248, as shown in FIG. 2, was used to fit the BRAVO-predicted incidence rates 174 to observed incidence rates 232, and slope, intercept, and R2 were used to show prediction 174 accuracy. As shown in FIG. 3, the microsimulation was conducted using a joint program 106 written through Visual Basic and C++ language. In addition, one-way sensitivity analyses were conducted to explore the impact of six risk factors 202 on the life expectancy in the ACCORD trial 254 population, shown in FIG. 4. The values of continuous risk factors 202 (HbA1c 126, SBP 128, LDL 132, and BMI 130) were set from one standard deviation below the mean to one standard deviation above, while categorical risk factors 202 (smoking 234 and severe hypoglycemia 156) ranged from 50% to 200% of the population average.

The structure of the simulation model is provided in FIG. 3. To reflect the chronic nature of diabetes, the model followed T2DM patients from any specified time point to death or a user specified time horizon. K cohorts were generated from the user defined distribution of the population characteristics, and i patients for each of the cohorts were then be simulated. For each patient 194 in the same cohort, demographic characteristics 190 and risk factors 202 were assigned based on the assigned cohorts' parameter values 164. Then, based on that information, the first year macrovascular 134, microvascular 146, adverse events 154 (e.g. hypoglycemia 156, 158) and mortality 178 for this patient 194 were predicted 174 through the risk equations 172. All of the events 134, 146, 154 were predicted 174 at a random order to account for event inter-dependency. After that, the comorbidities were updated for this patient 194 and his/her survival status 198 was checked. If this patient 194 was identified to be dead during this year, then the simulation for this patient 194 ended and the model moved on to the next patient 194 in the same cohort. If the patient 122 is not dead, risk factors 202 (i.e. HbA1c 126, SBP 128, weight 130, lipids 132) are updated based on the BRAVO risk engine 100 equations 172 and the simulation moved on to the next cycle and the same prediction process was conducted again. This iteration 112 was kept going until either the patient 194 was dead or the simulation reached its time horizon. And after all the patients 194 in this cohort had been simulated, the model moves on to the next cohort until all the cohorts were simulated. After simulation is completed, all of the outcomes were summarized, including incidence rates of each event type 134, 146, 154, mortality rates 178, life expectancy and etc.

The variable selection process was conducted through a mixed algorithm. Variables were included into the final model based on the following criteria:

C-statistic has been widely applied when selecting appropriate variables for regression models intended to predict 174 the risk of events 134, 146, 154, as it is a good measurement for the discrimination power of the model. However, in the BRAVO risk engine 100, the engine 100 includes discrimination power of the model, but also the prediction 174 accuracy. Thus, both c-statistic and Bier Score were calculated to support the model selection process. For logistic regression 244, the calculation process of c-statistic and Bier Score are straight forward. However, it became difficult in the survival regression framework, as it has multiple time periods. Two approaches were applied in this embodiment: 1) calculations for the five-year cumulative incidence as the predicted probability 174, and calculations of c-statistic and Bier Score based on five-year observed data 232 and, 2) calculations for time 170 dependent c-statistic and Bier Score, using formula suggested by Gerds et al and Potapov et al. For continuous variables as the outcome (e.g., HbA1c 126, SBP 128, LDL 132, BMI 130), the mean square error (MSE) was calculated.

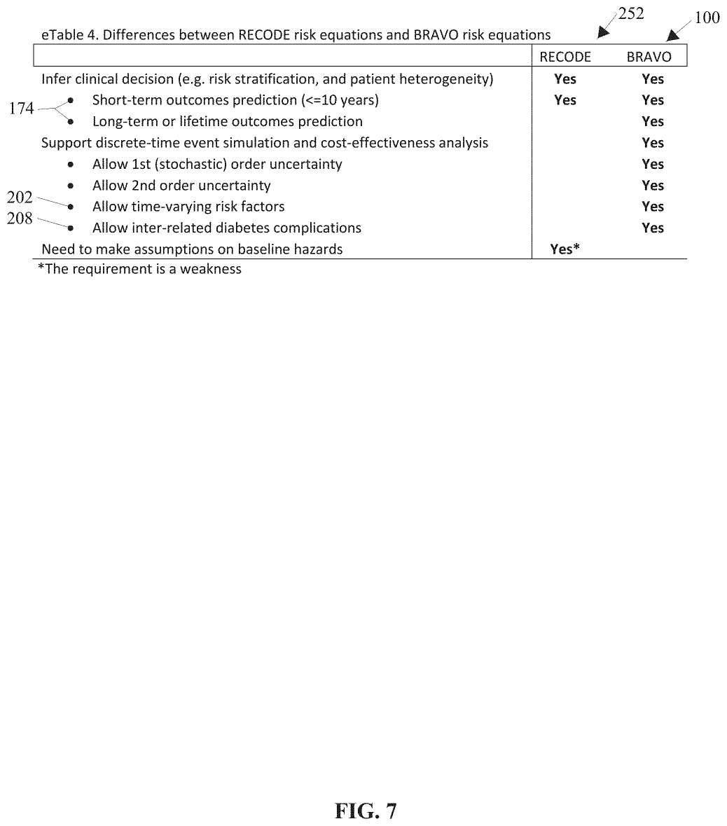

A recently published RECODe risk engine 252 has also used the ACCORD trial 254 data to develop a set of risk equations for modeling the risk of diabetes complications. Referring to FIG. 7, besides the methodological differences in the modeling strategy, outcomes inclusion, and variable definition, the BRAVO risk engine 100 and RECODe risk engine 252 have very different purposes of risk predictions 174. The RECODe risk engine 252 is intended for use in clinical settings to assist the initial treatment decision because the models only use baseline characteristics 162 to predict 174 the incidence rates of diabetes outcomes in a specified period (5 or 10 years). The BRAVO risk engine 100 aims to use an agent-based microsimulation modeling algorithm and to ultimately develop a diabetes model which is a predictor 174 for life-time disease progression 198, 202. Because the BRAVO risk engine 100 uses all time-dependent biomarkers 188, disease history 186, and other risk factors 202, the BRAVO risk engine 100 will be more appropriate than the RECODe risk engine to support a cost-effectiveness analysis such as prioritizing therapeutic strategies 196 or treatment targets of HbA1c 126, LDL 132, and blood pressure 128. A table has been developed to briefly summarize the key differences between the RECODe risk engine 252 and the BRAVO risk engine 100, as shown in FIG. 7.

Referring to FIGS. 11, 16, and 17, an analysis of the CHF 140 equation shows that having a history of MI 138 increased the risk of CHF 140 by 82.2%. In addition, repeat events were also taken into consideration. Having a history of CHF 140 was associated with a 247.6% increase in the risk 202 of a second CHF event 140.

The modeling approach of the present embodiment has also demonstrated a novel approach to using clinical trials with a limited length of follow-up time. While the ACCORD trial 254 only ran for 7 years, the ACCORD cohort 254 covered a wide range of diabetes durations, characteristics of ACCORD 254 are shown in FIGS. 4 and 13. Therefore, as illustrated in FIGS. 14-29 the BRAVO risk engine 100 used diabetes duration 168 as a time index 170 to simulate diabetes progression 198 and mortality 178 over 40 years. Further, indexing time 170 by diabetes duration 168 allowed us to estimate the time dependency of diabetes on events 134, 146, 154 and mortality 178.

Equations 172, illustrated in FIG. 2, from the BRAVO risk engine 100 have been validated internally and externally, as shown in FIG. 11. The external validation results, shown in FIG. 11, plot the observed incidence rates 232 against the predicted incidence rates 231. A simulation disease model based on the BRAVO risk engine 100 can be applied to predict 174 a range of long-term diabetes-related outcomes 176 to assist clinical 186 and policy decision making.

One having ordinary skill in the art will readily understand that the invention as discussed above may be practiced with steps in a different order, and/or with hardware elements in configurations that are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should also be made to the appended claims.

All U.S. patents and publications identified herein are incorporated in their entirety by reference thereto.

The claimed invention is:

1. A method of predicting diabetes progression and mortality comprising:
receiving a target population dataset comprising a series of baseline biological characteristics of the target population;
assigning parameter values based upon a user defined distribution of population characteristics;
analyzing, using a computer processor, the population dataset for generating a diabetes risk engine wherein diabetes duration is used as a time index, said diabetes risk engine operating one or more inter-correlated risk equations can be used as a predictor to determine a risk of a diabetes-related event or mortality of a patient;
adjusting the risk engine based upon new data collected based upon a new set of annual values collected during the generating of the population dataset;
receiving a first patient medical information comprising clinical, biomedical, and demographic factor information;
predicting a risk of an occurrence of one or more diabetes-related events with said diabetes risk engine based upon said parameter values, by comparing the first patient medical information to the population dataset;
providing at least one clinical or behavioral modification recommendation based on the predicted risk of the occurrence of said one or more diabetes-related events or mortality; and
tracking a progression and survival status of a user taking action toward achieving goals associated with improving health based upon the at least one clinical or behavioral modification recommendation.

2. The method of claim 1, further comprising a step of predicting a progression of risk factors of said first patient.

3. The method of claim 1, wherein the inter-correlated risk equations account for risk escalation as diabetes progress and during interactions between complications.

4. The method of claim 1, wherein said first patient medical information comprises risk factors comprising medication adherence, lifestyle modification, and therapy escalation.

5. The method of claim 1, wherein said diabetes-related event is a macrovascular event, wherein said macrovascular event is a stroke.

6. The method of claim 1, wherein said diabetes-related event is a macrovascular event, wherein said macrovascular event is myocardial infarction.

7. The method of claim 1, wherein said diabetes-related event is a macrovascular event, wherein said macrovascular event is congestive heart failure.

8. The method of claim 1, wherein said diabetes-related event is a macrovascular event, wherein said macrovascular event is angina.

9. The method of claim 1, wherein said diabetes-related event is a macrovascular event, wherein said macrovascular event is revascularization surgery.

10. The method of claim 1, wherein said diabetes-related event is a microvascular event, wherein said microvascular event is end stage renal failure.

11. The method of claim 1, wherein said diabetes-related event is a microvascular event, wherein said microvascular event is blindness.

12. The method of claim 1, wherein said diabetes-related event is a microvascular event, wherein said microvascular event is severe pressure sensation loss.

13. The method of claim 1, wherein said diabetes-related event is an adverse event, wherein said adverse event is severe hypoglycemia.

14. The method of claim 1, wherein said diabetes-related event is an adverse event, wherein said adverse event is symptomatic hypoglycemia.

15. A method of predicting an occurrence of one or more diabetes related events comprising:
receiving a target population dataset comprising a series of baseline characteristics of the target population;
assigning parameter values based upon a user defined distribution of population characteristics;
analyzing, using a computer processor, the population dataset for generating a diabetes risk engine wherein diabetes duration is used as a time index, said diabetes risk engine operating one or more inter-correlated risk equations configured to determine the risk of a diabetes-related event or mortality of the patient;
adjusting the risk engine based upon new data collected based upon a new set of annual values collected during the generating of the population dataset;
receiving a first patient medical information comprising clinical, biomedical, and demographic factor information from a first patient user;
predicting a risk of an occurrence of one or more diabetes-related events with said diabetes risk engine based upon said parameter values, by comparing the first patient medical information to the population dataset of said diabetes risk engine;
providing at least one clinical or behavioral modification recommendation based on the risk of the occurrence of said one or more diabetes-related events; and
tracking the survival status of a user taking action toward achieving goals associated with improving health based upon the at least one clinical or behavioral modification recommendation.

16. A diabetes risk engine system comprising:
at least one processor;
at least one memory unit containing computer program code;
a diabetes-related events module configured to predict an occurrence of one or more diabetes-related events through an iterative process;
a risk factors module to predict a progression of one or more risk factors through the iterative process;
a mortality module to predict an occurrence of mortality of a patient through the iterative process; and
a display interface configured to display the predicted risk of the one or more diabetes-related events.

17. The diabetes risk engine system of claim 16, wherein the risk factor is selected from the group consisting of glycosylated hemoglobin (HbA1c), systolic blood pressure (SBP), weight, and low-density lipoprotein cholesterol (LDL-C).

18. The diabetes risk engine system of claim 16, wherein said diabetes-related event is a macrovascular event, wherein said macrovascular event is a stroke.

19. The diabetes risk engine system of claim 16, wherein said diabetes-related event is a macrovascular event, wherein said macrovascular event is myocardial infarction.

20. The diabetes risk engine system of claim 16, wherein said diabetes-related event is a macrovascular event, wherein said macrovascular event is congestive heart failure.

21. The diabetes risk engine system of claim 16, wherein said diabetes-related event is a macrovascular event, wherein said macrovascular event is angina.

22. The diabetes risk engine system of claim 16, wherein said diabetes-related event is a macrovascular event, wherein said macrovascular event is revascularization surgery.

23. The diabetes risk engine system of claim 16, wherein said diabetes-related event is a microvascular event, wherein said microvascular event is end stage renal failure.

24. The diabetes risk engine system of claim 16, wherein said diabetes-related event is a microvascular event, wherein said microvascular event is blindness.

25. The diabetes risk engine system of claim 16, wherein said diabetes-related event is a microvascular event, wherein said microvascular event is severe pressure sensation loss.

26. The diabetes risk engine system of claim 16, wherein said diabetes-related event is an adverse event, wherein said adverse event is severe hypoglycemia.

27. The diabetes risk engine system of claim 16, wherein said diabetes-related event is an adverse event, wherein said adverse event is symptomatic hypoglycemia.
